(19) United States
(12) Patent Application Publication
(10) Pub. No.: US 2020/0062820 A1
(43) Pub. Date: Feb. 27, 2020

(54) Method for Treating Ischemic Tissue

(71) Applicant: UNIVERSITY OF MIAMI, Miami, FL (US)

(72) Inventors: Omaida Velazquez, Miami, FL (US); Zhao-Jun Liu, Miami, FL (US)

(21) Appl. No.: 16/610,029

(22) PCT Filed: May 2, 2018
(86) PCT No.: PCT/US2018/030615

§ 371 Date: Oct. 31, 2019

Related U.S. Application Data
(60) Provisional application No. 62/500,470, filed on May 2, 2017.

(51) Int. Cl. C07K 14/705; A61K 35/761; A61K 48/00; A61P 17/02; C12N 15/86; A01K 67/027

(52) U.S. Cl. CPC C07K 14/70564; A61K 35/761; A61K 48/005; A61P 17/02; C12N 15/86; A01K 2267/0375; A01K 2207/20; A01K 2227/105; C12N 2750/14143; A01K 2267/0337; A01K 67/0275

(57) ABSTRACT

The invention provides a method of increasing blood flow or perfusion in an ischemic tissue; inducing angiogenesis, neovascularization or revascularization; increasing skeletal muscle viability; promoting ischemic skin wound healing; treating or preventing gangrene; and/or treating CLI. In various aspects, the method comprises administering to a subject a hybrid adenoassociated virus (AAV) comprising a nucleotide sequence encoding an E-selectin, AAV serotype 2 (AAV2) inverted terminal repeats (ITRs), and a capsid from an AAV other than serotype 2. In various aspects, the method comprises administering to the subject a cell comprising an AAV comprising a nucleotide sequence encoding an E-selectin, AAV2 ITRs, and a AAV2 capsid.

### GRANT FUNDING DISCLOSURE

This invention was made with government support under grant number HHSN268201700008C, awarded by the National Institutes of Health/National Heart, Lung, and Blood Institutes. The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 38,503 byte ACII (Text) file named “51600A_SeqListing.txt”; created on May 1, 2018.

### FIELD OF DISCLOSURE

The disclosure relates to materials and methods for treating ischemic tissue.

### BACKGROUND OF THE INVENTION

Critical limb ischemia (CLI) is a severe blockage of the arteries that supply the limbs and represents an advanced stage of peripheral arterial disease (PAD) caused by systemic atherosclerosis. In atherosclerosis, fatty deposits (plaques) build up in the artery walls and make it difficult for blood to flow through the arteries. Atherosclerosis-associated obstruction of the arteries markedly reduces blood flow to the extremities (legs, feet and hands) and can cause severe pain, skin ulcers, sores, gangrene, and/or tissue loss. Many patients are at very high risk of major amputation and experience poor physical function and severely diminished quality of life. Particularly, CLI in diabetic patients is associated with high rates of morbidity and mortality.

Each year there are approximately 160,000 new patients who have CLI in the United States. Pharmacologic treatments have had limited impact on the outcome of PAD when the disease progresses to a state of CLI. The current standard therapy for CLI aimed at improving blood flow to the affected extremity is either surgical bypass of the clogged vessels or endovascular revascularization (reopening clogged vessels), including percutaneous transluminal angioplasty (PTA) and stent placement. However, approximately 20% to 40% of patients are not suitable for such interventions due to high operative risk, high recurrence of vessel blockage and graft failure rate or unfavorable endovascular anatomy, especially in the area below the knee. These patients often have ‘no-option’ other than amputation.

New effective strategies are needed to offer patients suffering from ischemia a viable therapeutic alternative.

### SUMMARY OF THE INVENTION

The disclosure provides a method of increasing blood flow or perfusion in an ischemic tissue in a subject. The method comprises administering to the subject in an amount effective to increase the blood flow or perfusion in the ischemic tissue a hybrid adenoassociated virus (AAV) comprising a nucleotide sequence encoding an E-selectin, AAV serotype 2 (AAV2) inverted terminal repeats (ITRs), and a capsid from an AAV other than serotype 2.

Also provided is a method of inducing angiogenesis, neovascularization or revascularization in an ischemic tissue in a subject, the method comprising administering to the subject in an amount effective to induce angiogenesis, neovascularization or revascularization in the ischemic tissue a hybrid AAV comprising a nucleotide sequence encoding an E-selectin, AAV2 ITRs, and a capsid from an AAV other than serotype 2.

The disclosure further provides a method of increasing skeletal muscle viability in a subject. The method comprises administering to the subject in an amount effective to increase the skeletal muscle viability a hybrid AAV comprising a nucleotide sequence encoding an E-selectin, AAV2 ITRs, and a capsid from an AAV other than serotype 2.

In addition, a method of promoting ischemic skin wound healing in a subject is provided. The method comprises administering to the subject in an amount effective to promote ischemic skin wound healing a hybrid AAV comprising a nucleotide sequence encoding an E-selectin, AAV2 ITRs, and a capsid from an AAV other than serotype 2.

The disclosure also is directed to a method of treating or preventing gangrene in a subject, the method comprising administering to the subject in an amount effective to treat or prevent gangrene in the subject a hybrid AAV comprising a nucleotide sequence encoding an E-selectin, AAV2 ITRs, and a capsid from an AAV other than serotype 2. Also provided is a method of treating critical limb ischemia (CLI) in a subject. The method comprises administering to the subject in an amount effective to treat CLI a hybrid AAV comprising a nucleotide sequence encoding an E-selectin, AAV2 ITRs, and a capsid from an AAV other than serotype 2.

The disclosure additionally provides a method of increasing blood flow or perfusion in an ischemic tissue in a subject, the method comprising administering to the subject a cell comprising an AAV comprising a nucleotide sequence encoding an E-selectin, AAV2 ITRs, and a AAV2 capsid in an amount effective to increase the blood flow or perfusion in the ischemic tissue.

Also provided is a method of inducing angiogenesis, neovascularization or revascularization in an ischemic tissue in a subject. The method comprises administering to the subject a cell comprising an AAV comprising a nucleotide sequence encoding an E-selectin, AAV2 ITRs, and an AAV2 capsid in an amount effective to induce angiogenesis, neovascularization or revascularization in the ischemic tissue.

A method of promoting ischemic skin wound healing in a subject is provided wherein the method comprises administering to the subject a cell comprising an AAV comprising a nucleotide sequence encoding an E-selectin, AAV2 ITRs, and a AAV2 capsid in an amount effective to promote ischemic skin wound healing. The disclosure also is directed to a method of treating or preventing gangrene in a subject, the method comprising administering to the subject a cell comprising an AAV comprising a nucleotide sequence encoding an E-selectin, AAV2 ITRs, and a AAV2 capsid in an amount effective to treat or prevent gangrene in the subject. Also provided is a method of treating critical limb ischemia (CLI) in a subject, wherein the method comprises administering to the subject a cell comprising an AAV comprising a nucleotide sequence encoding an E-selectin, AAV2 ITRs, and a AAV2 capsid in an amount effective to treat the CLI in the subject.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. In addition, the invention includes, as an additional aspect, all embodiments of the invention narrower in scope in any way than the variations specifically mentioned above. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF THE FIGURES

FIG. 1: Efficiency of various hybrid AAV in the infection of tissue cells in mouse ischemic limb tissue. 1×10⁹ vg of GFP/AAV2/2, GFP/AAV2/5, GFP/AAV2/8 and GFP/AAV2/9 were injected intramuscularly into medial semimembranous hamstring muscle in FVB mice which were underwent femoral artery ligation (n=3). Limb tissues were harvested in 7 days post AAV injection and subjected to immunofluorescence staining. Numbers of GFP+ cells per high power field (HPF, X 40; y-axis) were counted. AAV2/8 and AAV2/9 exhibited high efficiency in infecting tissue cells.

FIG. 2: E-selectin/AAV gene therapy improves skeleton muscle viability in ischemic limb. LacZ/AAV (bars on left; x-axis) vs E-selectin/AAV (bars on right; x-axis) Postoperative Faber Ischemia Score (y-axis). LacZ/AAV n=7; E-selectin/AAV n=10. Postoperative (POD) day 1 (LacZ/AAV=2.57, E-selectin/AAV=1.1): p=0.213; POD 2 (LacZ/AAV=2.71, E-selectin/AAV=1.4): p=0.253; POD 3 (LacZ/AAV=3, E-selectin/AAV=1.5): p=0.168; POD 7 (LacZ/AAV=3.86, E-selectin/AAV=1.9): *p=0.041; POD 14 (LacZ/AAV=5.29, E-selectin/AAV=2.4): **p=0.009.

FIG. 3: E-selectin/AAV gene therapy improves ischemic limb perfusion. LacZ/AAV vs E-selectin/AAV Laser Doppler Imaging (LDI) Perfusion Ratio. LacZ/AAV n=7; E-selectin/AAV n=10. Pre-surgery: p=0.659; Postoperative (POD) day 7: p=0.066; POD 14: *p=0.008.

FIG. 4: E-selectin/AAV gene therapy improves neovascularization and foot perfusion in gangrene foot. LacZ/AAV vs E-selectin/AAV live animal Dil perfusion mean intensity score (y-axis) for ligated and non-ligated limbs (x-axis). LacZ/AAV: n=5; E-selectin/AAV: n=5; Ligated limb (LacZ/AAV=22.1, E-selectin/AAV=43.86): *p<0.027; Unligated limb (LacZ/AAV=26.7, E-selectin/AAV=49.48): **p<0.005.

FIG. 5: Higher number of myofibrils/hpf (y-axis) in LacZ/AAV ligated vs E-selectin ligated limb (x-axis); *p<0.0001. Quantitative data of average number of myofibrils per high power field (HPF) demonstrate a significantly higher number of myofibrils in the E-selectin/AAV ligated limb as compared to LacZ ligated limb.

FIG. 6: Quantitative data demonstrating percent area of wound healed (y-axis) from POD 0-10 (x-axis). LacZ/AAV (bottom line): n=10; E-selectin/AAV (top line): n=11. POD 0: p<0.000127; POD1: 0.000441; POD2: p<0.000189; POD3-10: p<0.0001.

FIG. 7: Schematic illustration of a hybrid AAV comprising AAV2 replicase and AAV9 capsid.

FIG. 8: Schematic illustration of an AAV used for transducing cells of the present disclosure.

FIG. 9: Representative images of high-limb gangrene in L-NAME treated FVB mice. Ischemic limb with gangrene are boxed and toes where gangrene developed at POD1 and PODS are pointed by red arrow.

FIG. 10: Laser Doppler Images of mice treated with E-selectin/AAV2 (top row) or LacZ/AAV2 (bottom row) at the indicated time points (pre- and post-surgery, POD 7 and POD14).

FIG. 11: Images of LacZ/AAV-treated or E-selectin/AAV-treated mice with an unligated or ligated limb.

FIG. 12A and FIG. 12B: Microscopic Images of Hematoxylin and Eosin Staining of E-selectin/AAV ligated vs LacZ ligated limbs at 10× magnification.

FIG. 13: images of wounds at the indicated timepoints of mice treated with E-selectin/AAV or LacZ/AAV.

### DETAILED DESCRIPTION OF THE INVENTION

In various aspects, the disclosure relates to materials and methods for increasing blood flow or perfusion in an ischemic tissue in a subject; inducing angiogenesis, neovascularization or revascularization; increasing skeletal muscle viability in a subject (a subject optionally suffering from or at risk of suffering from ischemia, such as critical limb ischemia (CLI)); promoting ischemic skin wound healing in a subject; treating or preventing gangrene in a subject; and/or treating CLI in a subject. In various aspects, the method comprises administering to the subject an effective amount of a hybrid adenoassociated virus (AAV) comprising a nucleotide sequence encoding an E-selectin, AAV serotype 2 (AAV2) inverted terminal repeats (ITRs), and a capsid from an AAV other than serotype 2. In various aspects, the method comprises administering to the subject a cell comprising an AAV comprising a nucleotide sequence encoding an E-selectin, AAV2 ITRs, and a AAV2 capsid in an amount effective to achieve the desired biological response (i.e., induce angiogenesis, neovascularization or revascularization in the ischemic tissue, etc.). This disclosure is the first to show that virus-mediated production of E-selectin ameliorates symptoms of gangrene in vivo.

Aspects of the invention are described further below. The use of section headings are merely for the convenience of reading, and not intended to be limiting per se. The entire document is intended to be viewed as a unified disclosure, and it should be understood that all combinations of features described herein are contemplated.

### E-Selectin

E-selectin is a cell adhesion molecule typically expressed on endothelial cells. E-selection is also known as CD62 antigen-like family member E (CD62E), endothelial-leukocyte adhesion molecule 1 (ELAM-1), and leukocyte-endothelial cell adhesion molecule 2 (LECAM2). In various aspects, the E-selectin is native human E-selection. In this regard, the nucleic acid sequence encoding E-selectin is optionally a nucleic acid sequence encoding the human E-selectin protein (i.e., the E-selectin protein of SEQ ID NO: 1, which corresponds to Accession no. AAQ67702, NP_000441.2). In exemplary aspects, the nucleic acid sequence encodes the mature form of human E-selectin and does not contain a signal peptide MIASQFLSALTLVLLIKESGA (SEQ ID NO: 7). In exemplary aspects, the nucleic acid sequence encodes the mature form of human E-selectin of SEQ ID NO: 8. In various embodiments, the nucleic acid sequence encodes a protein that shares at least 65% (e.g., at least 75%, at least 80%, at least 85%, at least 90%, at least 95%, at least 96%, at least 97%, at least 98%, or 99%) amino acid sequence identity with SEQ ID NO: 1 and demonstrates at least one activity associated with native E-selectin, such as mediating EC-EPC adhesion or promoting accumulation of blood leukocytes at sites of inflammation. In various aspects, the nucleic acid sequence encoding E-selectin is set forth in SEQ ID NO: 2, which corresponds to Accession no. NM_000450. It will be appreciated that a nucleic acid encoding an allelic variant and homolog of human E-selectin is also contemplated. In various embodiments, the nucleic acid sequence is at least 65% (e.g., at least 75%, at least 80%, at least 85%, at least 90%, at least 95%, at least 96%, at least 97%, at least 98%, or 99%) identical to SEQ ID NO: 2. If desired, non-human, mammalian E-selectin also may be used; the amino acid sequence of mouse E-selectin (GenBank Accession No. AAA37577.1), rat E-selectin (GenBank Accession No. AAA41113.1), canine E-selectin (GenBank Accession No. AAA30843.1), and sheep E-selectin (GenBank Accession No. NP_001009749.1) are provided as SEQ ID NOs: 3-6, respectively.

As used herein, “at least 90% identity” and similar terms encompass any integer from, e.g., 90% to 100%, such as 90%, 91%, 92%, 93%, 94%, 95%, 96%, 97%, 98% or 99% and the like. Also, the term “at least [percentage] identity” encompasses any percentage that is greater than or equal to the number of identical nucleotides or amino acids divided by the total number of nucleotides or amino acids ([at least percentage identity]×[number of identical nucleotides or amino acids]/[total number of nucleotides or amino acids]). The calculation of percent identity of aligned amino acids (or nucleotides) of two or more sequences is well understood in the art and is determined conventionally using known computer programs. For example, alignment of two or more sequences to determine percent sequence identity is optionally performed using the algorithm described by Altschul et al. (Nucleic Acids Res., 25:3389-402 (1997)) as incorporated into BLAST (basic local alignment search tool) programs, available on the National Center for Biotechnology Information website.

Variant E-selectin proteins that differ from SEQ ID NO: 1 can be generated by making nucleotide substitutions that cause changes in the encoded polypeptide. Examples of substitutions are those that cause changes in (a) the structure of the polypeptide backbone; (b) the charge or hydrophobicity of the polypeptide; or (c) the bulk of an amino acid side chain. In various aspects, the variant E-selectin comprises one or more conservative substitutions, i.e., at least one amino acid of the protein is substituted with another amino acid having similar characteristics.

### Adeno-Associated Virus

In various embodiments, the method comprises administering to a subject an effective amount of a hybrid adenoassociated virus (AAV) comprising a nucleotide sequence encoding an E-selectin. By “hybrid AAV” is meant an AAV comprising portions of at least two AAV serotypes. In exemplary aspects, the hybrid AAV is not naturally-occurring and is engineered to comprise portions of AAV from two different AAV serotypes. “Hybrid AAV” are synonymous with AAV hybrid serotypes as described in Choi et al., Current Gene Ther 5(3): 299-310 (2005) and Wu et al., Mol Ther. 14(3):316-27 (2006). In exemplary aspects, the hybrid AAV comprises AAV2 ITRs in the viral genome, which is packaged in a capsid from an AAV other than serotype 2. The AAV mediates E-selectin production in target cells. In various aspects, the method comprises administering to the subject a cell comprising an AAV comprising viral genome comprising a nucleotide sequence encoding an E-selectin and AAV2 ITRs, which is packaged into an AAV2 capsid.

AAV is a DNA virus not known to cause human disease, making it a desirable gene therapy options. The AAV genome is comprised of two genes, rep and cap, flanked by inverted terminal repeats (ITRs), which contain recognition signals for DNA replication and viral packaging. AAV requires co-infection with a helper virus (i.e., an adenovirus or a herpes virus), or expression of helper genes, for efficient replication. AAV vectors used for administration of a therapeutic nucleic acid typically have a majority of the parental genome deleted, such that only the ITRs remain, although this is not required. Delivering the AAV rep protein enables integration of the AAV vector comprising AAV ITRs into a specific region of genome, if desired. Host cells comprising an integrated AAV genome show no change in cell growth or morphology. As such, prolonged expression of therapeutic factors from AAV vectors can be useful in treating persistent and chronic diseases. The AAV for use in the context of the disclosure is based on AAV type 2, and the viral genome delivered to the subject or cell comprises AAV2 ITRs. Other AAV serotypes include AAV type 1, AAV type 3 (including types 3A and 3B), AAV type 4, AAV type 5, AAV type 6, AAV type 7, AAV type 8, AAV type 9, AAV type 10, or AAV type 11. The genomic sequences of AAV, as well as the sequences of the ITRs, Rep proteins, and capsid subunits are known in the art. See, e.g., International Patent Publications Nos. WO 00/28061, WO 99/61601, WO 98/11244; as well as U.S. Pat. No. 6,156,303, Srivistava et al. (1983) J Virol. 45:555; Chiorini et al (1998) J Virol. 71:6823; Xiao et al (1999) J Virol. 73:3994; Shade et al (1986) J Virol. 58:921; and Gao et al (2002) Proc. Nat. Acad. Sci. USA 99:11854.

In various aspects, the AAV comprises a viral genome lacking all or part of the native AAV genome. For example, the AAV genome lacks all native AAV protein coding sequences, but retains the AAV ITRs (e.g., AAV2 ITRs), and further comprises the nucleic acid sequence encoding E-selectin.

The viral genome comprising the nucleic acid sequence and AAV2 ITRs can be incorporated into an virion (i.e., packaged into a viral capsid) to facilitate introduction of the genome into a cell. AAV capsid proteins compose the exterior, non-nucleic acid portion of the virion and are encoded by the AAV cap gene. The cap gene encodes three viral coat proteins, VP1, VP2 and VP3, which are required for virion assembly. The construction of AAV virions is described in, e.g., U.S. Pat. Nos. 5,173,414; 5,139,941; 5,863,541; 5,869,305; 6,057,152; and U.S. Pat. No. 6,376,237; Rabinowitz et al., J. Virol. 76:791-801, 2002; and Bowles et al., J. Virol. 77:423-432, 2003.

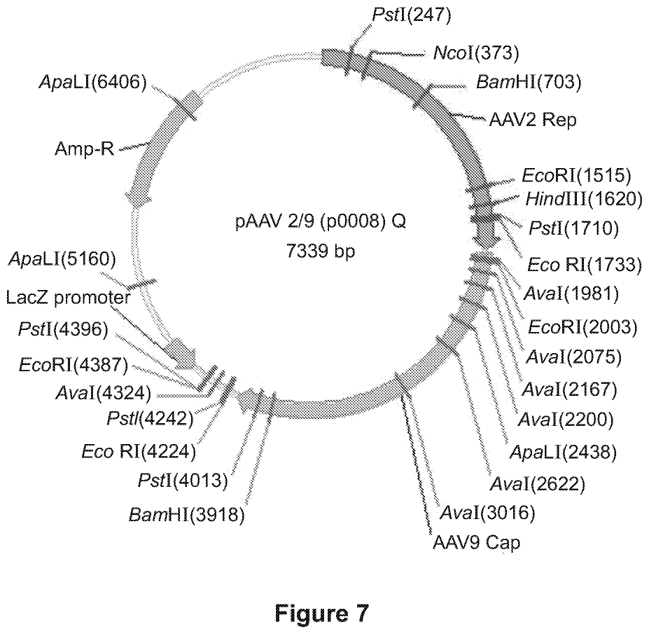

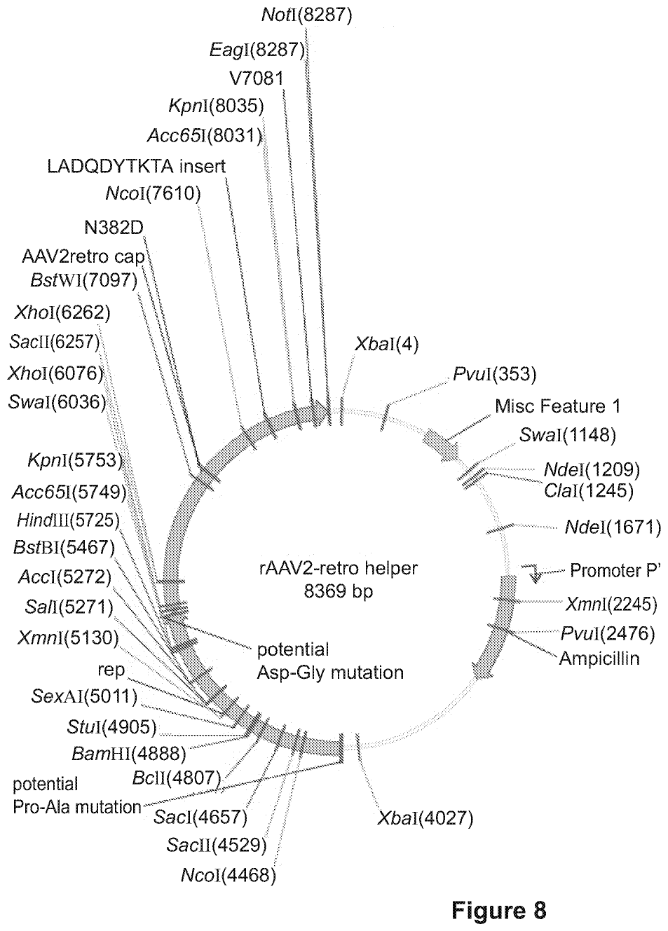

In various embodiments, the AAV genome comprising AAV2 ITRs is packaged into a capsid derived from a serotype other AAV2. Such AAV vectors are termed “pseudotyped” AAV or “hybrid” AAV. The AAV2 viral genome (comprising the nucleic acid sequence encoding E-selectin and AAV2 ITRs) is optionally packaged into a capsid from AAV type 1, AAV type 3 (including types 3A and 3B), AAV type 4, AAV type 5, AAV type 6, AAV type 7, AAV type 8, AAV type 9, AAV type 10, or AAV type 11. In various aspects, the AAV2 viral genome is packaged into an AAV8 capsid (AAV2/8) or AAV9 capsid (AAV2/9). Techniques involving the construction and use of pseudotyped AAV are further described in, e.g., Duan et al., J. Virol, 75:7662-7671, 2001; Halbert et al., J. Virol, 74:1524-1532, 2000; Zolotukhin et al, Methods, 28: 158-167, 2002; and Auricchio et al, Hum. Molec. Genet. 10:3075-3081, 2001. In exemplary aspects, the hybrid AAV of the disclosures comprises the elements shown in FIG. 7. In exemplary aspects, the hybrid AAV of the present disclosures comprises the structure shown in FIG. 7.

Optionally, the virus capsid (i.e., particle surface) is modified to adjust viral tropism. For example, components of the capsid can be modified to, e.g., expand the types of cells transduced by the resulting vector, avoid (in whole or in part) transduction of undesired cell types, or improve transduction efficiency of desired cell types (e.g., by incorporating a ligand for a cell surface receptor on desired cell type). Transduction efficiency is generally determined by reference to a control (i.e., an unmodified, matched viral vector). Improvements in transduction efficiency can result in, e.g., at least about 25%, 50%, 60%, 70%, 80%, 85%, 90%, 95%, 100% improvement in transduction rate of a given cell type. If desired, the capsid can be modified such that it does not efficiently transduce non-target tissues, such as liver or germ cells (e.g., 50% or less, 30% or less, 20% or less, 10% or less, 5% or less of the level of transduction of desired target tissue(s)). The construction and characterization of AAV capsid mutants including insertion mutants, alanine screening mutants, and epitope tag mutants is described in Wu et al, J. Virol. 74:8635-45, 2000. Other AAV that can be used in methods described herein include capsid hybrids that are generated by molecular breeding of viruses, as well as by exon shuffling. See Soong et al, Nat. Genet. 25:436-439, 2000; and Kolman and Stemmer Nat. Biotechnol 19:423-428, 2001.

Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001, all of which are hereby incorporated by reference, particularly with respect to the discussion of AAV production. Methods for using AAV vectors also are discussed, for example, in Tal, J., J. Biomed. Sci. 7:279-291, 2000 and Monahan and Samulski, Gene delivery 7:24-30, 2000.

Expression vectors, such as AAV vectors, typically contain a variety of nucleic acid sequences necessary for the transcription and translation of an operably linked coding sequence. For example, an expression vector can comprise origins of replication, polyadenylation signals, internal ribosome entry sites (IRES), promoters, enhancers, and the like. The AAV vector of the disclosure preferably comprises a promoter operably linked to the E-selectin coding sequence. “Operably linked” means that a control sequence, such as a promoter, is in a correct location and orientation in relation to another nucleic acid sequence to exert its effect (e.g., initiation of transcription) on the nucleic acid sequence. A promoter can be native or non-native to the nucleic acid sequence to which it is operably linked and native or non-native to a particular target cell type, and the promoter may be, in various aspects, a constitutive promoter, a tissue-specific promoter, or an inducible promoter. Examples of constitutive promoters include the Herpes Simplex virus (HSV), thymidine kinase (TK), Rous Sarcoma Virus (RSV), Simian Virus 40 (SV40), Mouse Mammary Tumor Virus (MMTV), Ad E1A, and cytomegalovirus (CMV) promoters. Examples of constitutive mammalian promoters include various housekeeping gene promoters, as exemplified by the β-actin promoter. Inducible promoters and/or regulatory elements are also contemplated for use in the methods described herein. Examples of inducible promoters include, but are not limited to, those from genes such as cytochrome P450 genes, heat shock protein genes, metallothionein genes, and hormone-inducible genes, such as the estrogen gene promoter. Another example of an inducible promoter is the tet promoter that is responsive to tetracycline. Tissue-specific promoters and/or regulatory elements are useful in certain embodiments of the methods described herein. Examples of such promoters include, but are not limited to, the Tie-2 or KDR promoter.

### Administration of Cells Producing E-Selectin

In some embodiments, the method comprises administering to the subject a cell comprising an AAV comprising a nucleotide sequence encoding an E-selectin and AAV2 ITRs. The AAV produces E-selectin in the cell. In various aspects, the AAV2 genome is packaged into an AAV2 capsid, although the AAV genome comprising AAV2 ITRs may be packaged in to a non-AAV2 capsid in various embodiments, as described further herein. The cell is, in various embodiments, a stem cell, such as a mesenchymal stem cell (MSC), a bone marrow (BM)-derived progenitor cell, or an endothelial progenitor cell (EPC). The cell may be isolated from the subject (i.e., autologous) or collected from a different donor (i.e., allogeneic). “Bone marrow-derived progenitor cells” and “BM-derived progenitor cells” mean progenitor cells that come from a bone marrow stem cell lineage. The cell also may be a mesenchymal stem cell (MSC), embryonic-like cells found in bone marrow that are capable of osteogenic, myogenic, adipogenic and chondrogenic differentiation.

In various aspects, the cell is an endothelial progenitor cell (EPC). By the “progenitor cell” or “endothelial progenitor cells” or “EPC” is meant any somatic cell which has the capacity to generate fully differentiated, functional progeny by differentiation and proliferation. In another embodiment, progenitor cells include progenitors from any tissue or organ system, including, but not limited to, blood, nerve, muscle, skin, gut, bone, kidney, liver, pancreas, thymus, and the like. Progenitor cells are distinguished from “differentiated cells,” which are cells which may or may not have the capacity to proliferate, i.e., self-replicate, but which are unable to undergo further differentiation to a different cell type under normal physiological conditions. Progenitor cells are further distinguished from abnormal cells such as cancer cells, especially leukemia cells, which proliferate (self-replicate) but which generally do not further differentiate, despite appearing to be immature or undifferentiated.

“Totipotent” cells are uncommitted progenitor cells, such as embryonic stem cells, i.e., both necessary and sufficient for generating all types of mature cells. Progenitor cells which retain a capacity to generate all pancreatic cell lineages but which cannot self-renew are termed “pluripotent.” In another embodiment, cells which can produce some but not all endothelial lineages and cannot self-renew are termed “multipotent.”

Techniques for isolating donor stem cells and transplantation of such isolated cells are known in the art. For example, target tissue or cells (e.g., BM-derived EPCs) are harvested from a host and exposed to the AAV virions described herein under conditions that promote infection, thereby introducing the E-selectin-encoding nucleic acid into the cells. These genetically modified cells are then be transplanted into the subject. Several approaches may be used for the introduction of cells into the subject, including intravenous injection, intraperitoneal injection, or in situ injection into target tissue. Microencapsulation of cells transduced or infected with AAV also is contemplated. Both autologous and allogeneic cell transplantation are contemplated in the context of the method of the disclosure.

Methods of Treatment; Uses

In various aspects, the disclosure relates to materials and methods for increasing blood flow or perfusion in an ischemic tissue in a subject. “Ischemic” refers to tissue that has become hypoxic (i.e., lacks sufficient oxygen), typically as a result of obstruction of the arterial blood supply or inadequate blood flow. In various aspects, the ischemic tissue is muscle tissue (skeletal muscle or cardiac muscle), although other tissues also are contemplated, such as retinal, adipose, liver, kidney, lung, gastrointestinal, pancreas, gall bladder, urinary bladder, central nervous tissue, and skin.

The AAV or cell is administered in an amount effective to increase the blood flow or perfusion in the ischemic tissue. It will be appreciated that any increase in perfusion or blood flow provides a benefit to the subject. Blood flow or perfusion in a tissue may be examined using, for example, Doppler imaging, positron emission tomography (PET), single photon emission computed tomography (SPECT), magnetic resonance imaging (MRI), or contrast-enhanced computed tomography (CT). For example, ⁹⁹mTc-sestamibi SPECT can be used to determine a summed stress score, which is a semiquantitative measure of perfusion obtained by summing the severity scores of hypoperfusion of 20 segments obtained by post-stress images. The severity scoring is defined as: 0=normal, 1=mildly reduced or equivocal, 2=moderately reduced, 3=severely reduced, and 4=absent uptake. Alternatively, perfusion may be characterized as the product of the mean velocity and the concentration of the red blood cells within the volume of the tissue being measured.

As used herein, the term “increase” and words stemming therefrom may not be a 100% or complete increase. Rather, there are varying degrees of an increase of which one of ordinary skill in the art recognizes as having a potential benefit or therapeutic effect. In this respect, the hybrid AAV or cells of the present disclosure may increase blood flow or perfusion to any amount or level. In exemplary embodiments, the increase provided by the methods of the present disclosure is at least or about a 10% increase (e.g., at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase).

Methods of measuring blood flow or perfusion are known in the art. Ischemic limb perfusion can be measured by, for example, Laser Doppler imaging (LDI), such as the LDI described herein in EXAMPLES. Other assays for measuring blood flow or perfusion include plethysmogrpahy, dye or thermal diffusion, contrast ultrasound, PET imaging, electromagnetic flow probes, video microscopy, near-infrared spectroscopy, labeled microspheres, microdialysis, diffuse correlation spectroscopy (DCS), arterial spin-labelled MRI, xenon-CT and Doppler ultrasound, which is described in Mesquita et al., Philos Trans A Math Phys Eng Sci. 369(1955): 4390-4406 (2011) and Barrett and Rattigan, Diabetes. 61(11): 2661-2668 (2012).

In various aspects, the disclosure relates to materials and methods for inducing angiogenesis, neovascularization, or revascularization in ischemic tissue. Neovascularization is the formation of new blood vessels. “Revascularization” is the restoration of perfusion to a body part or organ that has suffered ischemia. Angiogenesis is the growth of new blood vessels originating from existing blood vessels (i.e., growth of capillary buds from pre-existing blood vessels). Angiogenesis refers, in at least one embodiment, to the process by which resident endothelial cells of a wound's adjacent mature vascular network proliferate, and in other embodiments migrate, and remodel into neovessels that grow into the initially avascular wound tissue. “Inducing” angiogenesis, neovascularization, or revascularization includes aiding in the formation and/or quality of new blood vessels. If angiogenesis, neovascularization, or revascularization is not occurring, the angiogenesis, neovascularization, or revascularization can be initiated; if angiogenesis, neovascularization, or revascularization is already occurring, the angiogenesis, neovascularization, or revascularization can be enhanced or heightened. In this aspect, the AAV or cell is administered in an amount effective to induce angiogenesis, neovascularization, or revascularization. Angiogenesis or neovascularization can be detected and/or characterized by measuring the number of non-branching blood vessel segments (number of segments per unit area), the functional vascular density (total length of perfused blood vessel per unit area), and/or the vessel volume density (total of calculated blood vessel volume based on length and diameter of each segment per unit area).

Various aspects of the method can be used to deliver E-selectin to a variety of tissues, including, for example, skeletal muscle and cardiac tissue. The method can be used for the research or treatment of numerous diseases and ailments. For example, a method of promoting neovascularization or angiogenesis in ischemic tissue can be used to study or treat (therapeutically or prophylactically) peripheral vascular disease, mesenteric ischemia, cerebrovascular ischemia, muscle wasting due to ischemia, or complications associated with surgical procedures (e.g., healing or reattachment of skin and/or muscle flaps).

In various aspects, the disclosure relates to materials and methods for increasing skeletal muscle viability in a subject (e.g., a subject optionally suffering from or at risk of suffering from ischemia, such as critical limb ischemia (CLI)). The hybrid AAV or cells of the present disclosure may increase skeletal muscle viability to any amount or level. In exemplary embodiments, the increase provided by the methods of the present disclosure is at least or about a 10% increase (e.g., at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase). Methods of measuring skeletal muscle viability are known in the art and include counting the number of muscle cells in an Haemotoxylin and Eosin (H&E) stained slide (per high power field). See the EXAMPLES below.

In various aspects, the disclosure relates to materials and methods for promoting ischemic skin wound healing in a subject. “Promoting ischemic skin wound healing” encompasses, in various aspects of the disclosure, reducing the size of the wound, resolution of inflammation, inhibition of formation of necrotic tissue, repair of the underlying skin matrix, and re-epithelialization. Wounds and progression of wound healing can be determined by microscopy and examination of photographs taken over a period of time. LDI is an exemplary method of measuring ischemic wound healing. Image analyzers useful for studying wounds are available (e.g., AlphaEase FC version 4.1.0, Alpha Innotech Corporation). In various aspects, the method promotes wound re-epithelialization.

In various aspects, the disclosure relates to materials and methods for treating or preventing gangrene in a subject. Gangrene is a type of necrosis caused by insufficient blood supply. Gangrene can occur as a result of injury, infection, or chronic condition that negatively impacts blood circulation. Subjects at risk of gangrene include, but are not limited to, subjects suffering from infection, diabetes, circulatory/blood vessel diseases, or severe injury. Different types of gangrene are classified based on symptoms, and include, e.g., dry gangrene (characterized by dry, shriveled, discolored skin), wet gangrene (associated with bacterial infection, and often with swelling or blisters), gas gangrene (typically affecting deep muscle tissue and resulting in gas blisters), internal gangrene (affecting one or more of organs), and necrotizing fasciitis (caused by flesh-eating microbes). In various aspects, the gangrene is present in an extremity (e.g., toe, foot, leg, finger, or arm). Incidence and progression of gangrene may be assessed by visual inspection, photography, X-ray, computerized tomography (CT), magnetic resonance imaging (MRI), arteriogram (or other imaging assay used to visualize blood vessels), and/or tissue culture or biopsy. Gangrene also may be characterized using a modified Tarlov ischemia scale, wherein Laser Doppler Imaging (LDI) is optionally employed.

In various aspects, the disclosure relates to materials and methods for treating CLI in a subject. Critical limb ischemia arises from, e.g., the inability of arteries to conduct sufficient blood flow to the lower leg, ankle and toes. CLI can cause persistent, recurring rest pain (e.g., burning pain in the ball of the foot and toes), ulcers, and gangrene. CLI is marked by, for example, low or a lack of pulse in the foot, low ankle brachial index (ABI, blood pressure in ankle <0.4), reduced blood pressure in toe (<30 mm Hg), reduced transcutaneous oxygen, and/or muscle wasting. CLI is assessed using any of a number of ways, including (but not limited to) Doppler imaging, blood pressure cuff, flourescein angiography, TCOM (transcutaneous oxygen measurement), and/or functional assessments (muscle strength, walking tests, pain evaluations). The method, in various embodiments, improves any one or more of the CLI parameters described herein.

Efficacy in treating (i.e., reducing, easing, suppressing, or alleviating) or preventing a disorder or condition in a subject in need thereof is determined using any suitable method, including the methods described above. “Treatment” does not require a 100% abolition of a disorder in the subject. Any decrease in symptoms constitutes a beneficial biological effect in a subject. In various aspects, the method reduces severity (which can include reducing need for and/or amount of (e.g., exposure to) other drugs and/or therapies generally used for these conditions), duration, and/or frequency of pain. “Prevention” does not require a complete preclusion of the onset of a disorder or condition; any dampening or delay of the onset of a disorder or associated symptoms is contemplated.

For any of the parameters described herein, a therapeutic effect resulting from the method can be ascertained by, e.g., comparing baseline values to follow-up values. By “baseline values” is meant the values determined for each parameter performed in the baseline study recorded prior to treatment in accordance with the method. By “follow-up values” is meant the values determined for the same parameter(s) as in the baseline study recorded at an appropriate time after treatment (e.g., 1 week, 6 weeks, 12 weeks, 26 weeks, 36 weeks, 48 weeks, or 52 weeks post-treatment). Typically, multiple follow-up assessments are performed, and, thus, multiple follow-up values for the same parameters are ascertained at different time points post-treatment.

The disclosure further provides use of an AAV vector comprising AAV2 ITRs and a nucleic acid sequence encoding E-selectin in increasing blood flow or perfusion in an ischemic tissue in a subject; inducing angiogenesis, neovascularization or revascularization; increasing skeletal muscle viability in a subject (a subject optionally suffering from or at risk of suffering from ischemia, such as critical limb ischemia (CLI)); promoting ischemic skin wound healing in a subject; treating or preventing gangrene in a subject; and/or in the treatment of CLI in a subject. Also provided is use of an AAV vector comprising AAV2 ITRs and a nucleic acid sequence encoding E-selectin in the preparation of a medicament for increasing blood flow or perfusion in an ischemic tissue in a subject; inducing angiogenesis, neovascularization or revascularization; increasing skeletal muscle viability in a subject (a subject optionally suffering from or at risk of suffering from ischemia, such as critical limb ischemia (CLI)); promoting ischemic skin wound healing in a subject; treating or preventing gangrene in a subject; and/or treating CLI in a subject.

Also provided is use of a cell comprising an AAV comprising AAV2 ITRs and a nucleic acid sequence encoding E-selectin, packaged into an AAV2 capsid (or pseudotyped) in increasing blood flow or perfusion in an ischemic tissue in a subject; inducing angiogenesis, neovascularization or revascularization; increasing skeletal muscle viability in a subject (a subject optionally suffering from or at risk of suffering from ischemia, such as critical limb ischemia (CLI)); promoting ischemic skin wound healing in a subject; treating or preventing gangrene in a subject; and/or in the treatment of CLI in a subject. Also provided is use of a cell comprising an AAV comprising AAV2 ITRs and a nucleic acid sequence encoding E-selectin, packaged into an AAV2 capsid (or pseudotyped), in the preparation of a medicament for increasing blood flow or perfusion in an ischemic tissue in a subject; inducing angiogenesis, neovascularization or revascularization; increasing skeletal muscle viability in a subject (a subject optionally suffering from or at risk of suffering from ischemia, such as critical limb ischemia (CLI)); promoting ischemic skin wound healing in a subject; treating or preventing gangrene in a subject; and/or treating CLI in a subject.

The subject of the method of disclosure may be a mammal, such as a human, rat, mouse, cat, dog, goat, sheep, horse, monkey, ape, rabbit, cow, etc. The subject (e.g., mammal) can be in any stage of development, including adults or juveniles. Preferably, the subject is a human. The methods described herein generally include administration of a therapeutically effective amount of the compositions described herein to a subject (e.g., animal, human) in need thereof. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider. In various aspects, the subject has (or is at risk of) critical limb ischemia (CLI). In various aspects, the subject has (or is at risk of) peripheral artery disease (PAD). PAD is characterized by a narrowing of the peripheral arteries to the legs, stomach, arms, and head, often caused by plaque composed of, e.g., fat, cholesterol, fibrous tissue, calcium, and other blood components. Common symptoms of PAD include, but are not limited to, claudication (leg pain when walking), extremity weakness, cold sensation in the extremities, and discoloration. PAD is evaluated using, e.g., Doppler ultrasound and angiography.

### Formulations, Dose, Administration Regimens

In various aspects, the AAV or the cell is provided in a composition (e.g., a pharmaceutical composition) comprising a physiologically-acceptable (i.e., pharmacologically-acceptable) carrier, buffer, excipient, or diluent. Any suitable physiologically-acceptable (e.g., pharmaceutically acceptable) carrier can be used within the context of the disclosure, and such carriers are well known in the art. The choice of carrier will be determined, in part, by the particular site to which the composition is to be administered and the particular method used to administer the composition. The composition also can comprise agents which, for instance, facilitate uptake of the AAV into host cells. Suitable composition formulations include aqueous and non-aqueous solutions, isotonic sterile solutions, which can contain anti-oxidants, buffers, bacteriostats, and solutes that render the formulation isotonic with the blood, and aqueous and non-aqueous sterile suspensions that can include suspending agents, solubilizers, thickening agents, stabilizers, and preservatives.

The composition may be formulated for topical administration (e.g., in the form of aerosol, cream, foam, gel, liquid, ointment, paste, powder, shampoo, spray, patch, disk, or dressing). A “patch” typically includes at least the compositions provided herein and a covering layer, such that, the patch can be placed over an area of skin to be treated. The patch can be designed to maximize delivery of the compositions provided herein through the stratum corneum and into the epidermis or dermis, reduce lag time, promote uniform absorption, and reduce mechanical rub-off.

The composition can be presented in unit-dose or multi-dose sealed containers, such as ampules and vials, and can be stored in a freeze-dried (lyophilized) condition requiring only the addition of the sterile liquid carrier, for example, water, immediately prior to use. A composition comprising AAV or cells comprising AAV is, in one aspect, placed within containers, along with packaging material that provides instructions regarding the use of the composition (i.e., in a kit). Generally, such instructions include a tangible expression describing the reagent concentration, as well as, in certain embodiments, relative amounts of excipient ingredients or diluents (e.g., water, saline or PBS) that may be necessary to reconstitute the composition.

The AAV or cell is administered in an amount and at a location sufficient to provide some improvement or benefit to the subject, e.g., increase blood flow or perfusion in an ischemic tissue; induce angiogenesis, neovascularization or revascularization; increase skeletal muscle viability; promote ischemic skin wound healing; treat or prevent gangrene; and/or treat CLI. Depending on the circumstances, a composition comprising the AAV or cell is applied or instilled into body cavities, applied directly to target tissue, and/or introduced into circulation. For example, in various circumstances, it will be desirable to deliver the composition by intravenous, intraperitoneal, intracerebral (intra-parenchymal), intramuscular, intra-ocular, intraarterial, intraportal, intralesional, intramedullary, intrathecal, intraventricular, intradermal, intraarticular, intraneuronal, intraganglion, periganglion, transdermal, subcutaneous, intranasal, inhalation (e.g., upper and/or lower airways), enteral, epidural, urethral, vaginal, or rectal means. If desired, the AAV or cell is administered regionally via intramuscular, transdermal, or subcutaneous administration, or intraarterial or intravenous administration feeding the region of interest. In a preferred embodiment, the AAV or cell is intramuscularly administered to ischemic tissue in skeletal muscle.

Alternatively, the composition is administered locally via implantation of a membrane, sponge, capsule, or another appropriate material onto which the composition has been absorbed or encapsulated. Where an implantation device is used, the device is, in one aspect, implanted into a suitable tissue, and delivery of the AAV or E-selectin produced by the engineered cell is, for example, via diffusion, timed-release bolus, or continuous administration.

A particular administration regimen for a particular subject will depend, in part, upon the amount of therapeutic administered, the route of administration, and the cause and extent of any side effects. The amount administered to a subject (e.g., a mammal, such as a human) in accordance with the disclosure should be sufficient to affect the desired response over a reasonable time frame.

Exemplary doses of viral particles in genomic equivalent titers of 10⁴-10¹⁵ transducing units (e.g., 10⁷-10¹² transducing units), or at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, or at least about 10¹⁵ transducing units (e.g., at least about 10⁷, at least about 10⁸, at least about 10⁹, at least about 10¹⁰, at least about 10¹¹, at least about 10¹², at least about 10¹³ or at least about 10¹⁴ transducing units, such as about 10¹⁰ or 10¹² transducing units). In exemplary aspects, the dose of viral particles (VP) per in vitro transduced cell is within about 10³ to about 10¹². In some aspects, the dose of viral particles per in vitro transduced cell is within about 10⁴ to about 10⁸ or about 10⁴ to about 10⁶. For example, the dose of viral particles per in vitro transduced cell is 10⁵ VP/cell. Some conditions require prolonged treatment, which may or may not entail multiple administrations over time.

In exemplary aspects, the dose of the hybrid AAV administered to the subject (e.g., via intramuscular injection) is about 50 to about 5000 μl hybrid AAV, wherein the concentration of the hybrid AAV is within about 10⁸ or 10¹⁶ VP/ml. In exemplary aspects, the dose of the hybrid AAV administered to the subject (e.g., via intramuscular injection) is about 50 to about 500 μl hybrid AAV, wherein the concentration of the hybrid AAV is within about 10¹⁰ or 10¹⁴ VP/ml. In exemplary aspects, the dose of the hybrid AAV administered to the subject (e.g., via intramuscular injection) is about 75 to about 200 μl hybrid AAV, wherein the concentration of the hybrid AAV is about 10¹² VP/ml.

When appropriate, the AAV or cell is administered in combination with other substances (e.g., therapeutics) and/or other therapeutic modalities to achieve an additional (or augmented) biological effect. This aspect includes concurrent administration (i.e., substantially simultaneous administration) and non-concurrent administration (i.e., administration at different times, in any order, whether overlapping or not) of the AAV or cell and one or more additionally suitable agents(s). It will be appreciated that different components are, in certain aspects, administered in the same or in separate compositions, and by the same or different routes of administration.

According to a further aspect of the disclosure, the AAV or cell is optionally administered separately, sequentially or simultaneously in combination with one or more agents useful for treating the symptoms or causes of the ischemia. Representative agents include, but are not limited to, aspirin, nitrates, beta blockers, calcium channel blockers, cholesterol-lowering medications, angiotensin-converting enzyme (ACE) inhibitors, ranolazine, anticoagulants, thrombolytic agents (tissue plasminogen activator (tPA), streptokinase, or urokinase), antibiotics.

According to a further aspect of the disclosure, the AAV or cell is optionally administered separately, sequentially or simultaneously in combination with one or more agents useful for pain management. Examples of further agents include, but are not limited to, an opioid analgesic (e.g., morphine, hydromorphone, oxymorphone, fentanyl, codeine, dihydrocodeine, oxycodone, or hydrocodone); a nonsteroidal antiinflammatory drug (NSAID) (e.g., aspirin, diclofenac, ibuprofen, naproxen, oxaprozin, or cyclooxygenase-2 (COX-2) inhibitor); a sedative (e.g., a barbiturate sedative); an anesthetic; and a corticosteroid (e.g., dexamethasone).

In instances where it is desired to promote recruitment of BM-derived stem cells from the BM to a non-BM compartment (e.g., a target tissue), an agent capable of promoting recruitment of BM-derived progenitor cells also is provided to the subject, either as part of the composition or separate as part of a treatment regimen. A number of such agents are known and include, e.g., integrins, the selectin family of adhesion molecules, VCAM-I, and colony stimulating factors. Suitable agents are further described in, e.g., International Patent Publication WO 00/50048.

The method of the disclosure is optionally part of a treatment regimen that includes hyperbaric oxygen therapy (HBO₂), an adjunctive therapy used to stimulate wound healing in situations where the microvasculature has become attenuated. Methods of treatment involving HBO₂ are described in, for example, International Patent Application No. PCT/US2008/003760, which is incorporated herein by reference with respect to its discussion of hyperbaric oxygen therapy. Optionally, patients receive 20 or more treatments breathing 100% 02 in a pressurized chamber at between about 2.0 to about 3.2 atmospheres absolute (ATA), once or twice daily. Treatment time ranges are generally from about 10 minutes to about 240 minutes (e.g., about 10, 15, 30, 60, 90, 120, 150, 180, 210, 240, etc. minutes).

The treatment regimen may also comprise endovascular treatments (e.g., angioplasty or stenting), bypass or arterial surgery, or debridement.

The invention, thus generally described, will be understood more readily by reference to the following example, which is provided by way of illustration and is not intended to limit the invention.

### EXAMPLES

### Example 1

Impaired blood flow to the limb and soft tissue, which is normally through both macrovascular (arteries) and microvascular (capillaries) processes, is a central common etiology in patients suffering with PAD and CLI. The restoration of sufficient blood flow to ischemic tissue allows a successful repair response. Therapeutic angiogenesis refers to the use of drugs, genes, cells or mechanical devices to induce blood vessel formation in ischemic tissue. The primary benefit is inducing the growth of new blood vessels and promoting collateral vessel formation to increase blood flow to blood starved tissues. Angiogenesis can ultimately lead to a reduction in the risk of adverse cardiovascular events, relieve ischemic pain, heal ulcers, prevent major amputation, and improve quality of life and survival in CLI patients, particularly those who do not qualify for surgical intervention.

Adhesion molecules on the cell surface mediate cell-cell interaction and homing. Adhesion receptor/ligand pair (specifically E-selectin/ligands)-mediated cell-cell interaction between stem/progenitor cells and EC in injury tissue is an essential event in stem cell-induced blood vessel production. A novel strategy to treat PAD/CLI was tested by creating a supportive tissue microenvironment by priming EC in wound vasculature and tissue cells with E-selectin using gene-therapy (E-selectin/AAV). E-selectin can serve as docking site for either endogenous or exogenous of bone marrow (BM)-derived, repair-competent stem/progenitor tissue repair cells (TRC) (which express ligand of E-selectin) to anchor, by which to increase precision interaction/homing of TRC to ischemic tissue. The feasibility and efficacy of adhesion molecule-based extracellular and cellular components in mouse limb ischemia and gangrene models was tested, and it was demonstrated that E-selectin/AAV gene therapy is an effective modality for PAD/CLI in mouse model. The methods described herein improve the hospitability of the tissue microenvironment to enhance precision targeting of bone marrow (BM)-derived, repair-competent stem/progenitor cells (TRC).

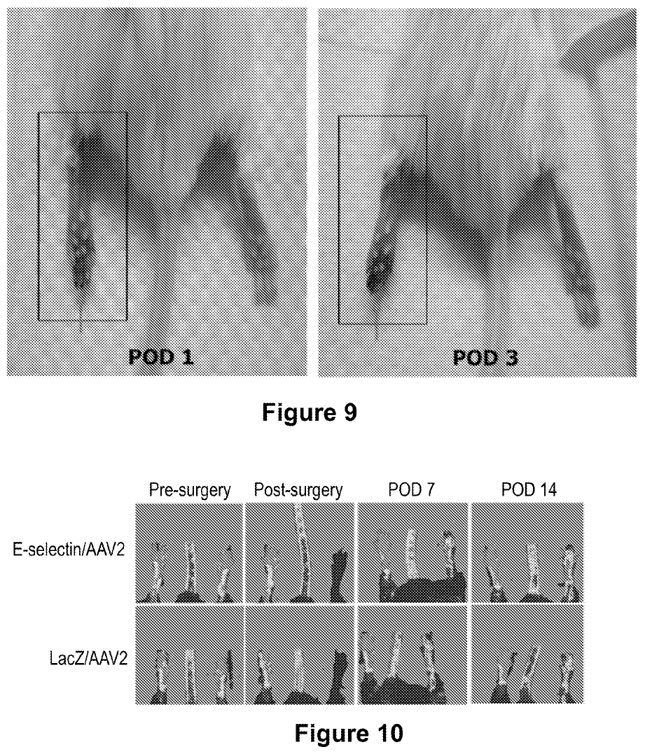

Gangrene is a particular type of tissue necrosis yet the underpinning molecular mechanism remains largely unknown. Lack of reliable and reproducible animal model of gangrene has made it difficult to study this disease process. As nitric oxide (NO) is known to act as a regulator of cellular response in oxidative conditions which is a hallmark of ischemia, the potential involvement of NO/NOS pathway in the development and progression of gangrene was investigated, and a murine model of limb gangrene was developed to delineate the molecular mechanisms involved in pathophysiological process of ischemia-induced limb gangrene. Eight to 12-week old male FVB mice were treated with N-Nitro-L-Arginine Methyl Ester (L-NAME) a non-selective NOS inhibitor and vehicle (n=18/group), respectively. Hindlimb ischemia was generated by ligation of the femoral artery at the inguinal ligament and popliteal fossa, followed by excision of the artery and all branches in right limb. Mice received a single dose of L-NAME (40 mg/kg ip) 1-2 hours pre-operatively, and a daily dose thereafter on post-operative day 1, 2 and 3 (40 mg/kg ip). Control mice received the medication vehicle alone. Incidence and progression of gangrene were assessed daily by visual inspection and photography. Gangrene was assessed using a modified Tarlov ischemia scale. Ischemia was monitored daily using Laser Doppler Imaging (LDI) from post-operative day (POD) 1 to 3. L-NAME treated mice uniformly developed distal limb gangrene by POD 3. In comparison, none of the mice in the control group developed gangrene (FIG. 9). This is the first reliable and high-reproducible murine ischemia-induced hindlimb gangrene model.

Recombinant AAV (rAAV) vectors are derived from a wild-type virus, AAV, which is non-pathogenic. No apparent ill effects have been associated with AAV even though the majority of humans have been exposed to this virus. Optionally, recombinant vectors used in the methods described herein are devoid of all AAV genes, that is, the rep and cap gene of the wild-type virus have been removed. The inverse terminal repeats (ITRs) are the only viral DNA sequences retained in the recombinant vector genome. In addition to its safety profile (the lack of pathogenicity and toxicity), recombinant AAV vector has the following prominent features; ability to infect dividing and non-dividing cells of various tissue origins, a very low host immune response and long-term expression.

The AAV life cycle is regulated through a complicated system involving host factors, helper virus, genes encoded in the AAV genome, and cis element ITRs. ITRs are palindromic sequences which can assume a T-shaped hairpin structure. This special configuration serves as the origin for viral DNA replication. In addition, the ITRs are essential for successful virus packaging, integration and rescue. The recombinant rAAV genome consists usually of single-stranded DNA as the genome of wild-type AAV. A deletion in the D-region of one of the ITRs of the proviral plasmids leads to efficient packaging of double-stranded rAAV, which are usually referred to as ‘self-complementary’ rAAV. The self-complementary genomes of rAAV can be packaged in certain capsids to determine their tropism. AAV can be ‘packaged’ in capsids of many different serotypes to “pseudotype” the AAV vector, whereby an expression cassette based on the genome of AAV (that is, the origin of the ITRs) is packaged into a recombinant viral particle with a capsid originating from another serotype of AAV. The pseudotyped recombinant AAV vectors are often designated rAAV2/1 or rAAV2/9, and so on, referring to their hybrid origin (genome based on AAV2 packaged in AAV1, or AAV9 capsid). Pseudotyped AAV vectors mediate differing patterns and kinetics of transgene expression, which considerably expands the available repertoire of AAV vectors.

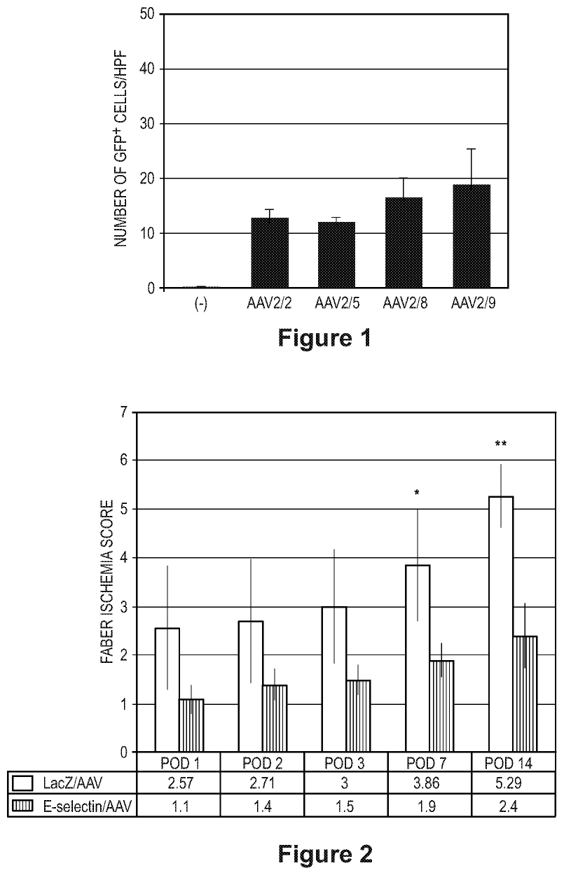

The efficiency of different AAV vectors encoding GFP has been tested, including, AAV2/2, AAV2/5, AAV2/8 and AAV2/9, for limb ischemia in mouse model. AAV2/9 exhibited higher transduction efficiency (FIG. 1). The basic AAV2 cis-plasmid are pZac based. pZac contains two AAV ITRs at the two ends, the CMV promoter, the multiple cloning site (MCS) and SV40 polyA for easy cloning in the gene of interest. Murine E-selectin/AAV2/9 (LacZ/AAV2/9 as control) was used to transduce ischemic limb tissue to prime the ischemic tissue microenvironment in a mouse hindlimb gangrene model. E-selectin/AAV2/9 and LacZ/AAV2/9 viruses were injected intramuscularly (1.8×10¹² vg and 1.2×10¹³ vg, respectively. Five sites of injection of 20 μL each were performed along the semimembranous hamstring muscle in the thigh) into the ischemic limb of gangrene mouse, respectively. Tissue cell expression of transgene (E-selectin) was examined by immunohistochemistry. Therapeutic effects of E-selectin/AAV2/9 gene therapy in treatment of gangrene were assessed by monitoring hindlimb blood flow using Laser Doppler Imaging (LDI) and measuring neovascularization and revascularization using blood vessel imaging technology.

Eight to twelve week old FVB/NJ male mice were used for priming bilateral hindlimb tissue microenvironment with intramuscular E-selectin/AAV 1.8×10¹² vg (treatment group) or LacZ/AAV2/9 1.2×10¹³ vg (control group) in 20 μL increments 5× along medial semimembranous hamstring muscle. Priming was performed 4, 2 and 0 days prior to hindlimb surgery. FVB mice underwent combined femoral artery ligation/excision and administration of NG-nitro-L-arginine methyl ester (L-NAME), nitric oxide synthase inhibitor, which further reduces hindlimb perfusion, 30 minutes prior to surgery and on postoperative days 1, 2 and 3. Ischemia score based on the Faber scale for gangrene severity was recorded on postoperative days 1, 2, 3, 7 and 14, whereas laser Doppler imaging was performed on postoperative days 7 and 14. Live animal dil perfusion with laser scanning confocal microscopy to quantify neovascularization was performed on postoperative day 14, at which point the animal was sacrificed and thigh tissue harvested for immunofluorescence to verify E-selectin transgene expression.

All mice underwent femoral artery ligation in addition to L-NAME administration. Tissue was assigned a score based on the Faber ischemia scoring system (a scale of 0 (no gangrene) to 11 (severe forefoot gangrene)). Faber Ischemia Scoring is described in Faber et al., Arteriosclerosis, thrombosis, and vascular biology 31(8):1748-1756 (2011). Based on the Faber ischemia scoring scale of 0 (no gangrene) to 11 (severe forefoot gangrene), by postoperative day 7 all mice were found to have presence of gangrene. Severity of gangrene was significantly worse in the control group having received LacZ/AAV by postoperative day 7 as compared to the treatment group having received E-selectin/AAV. This significance was even more profound by postoperative 14 where either aforementioned group had an ischemia score of 5.3 and 2.4, respectively (p<0.05, FIG. 2). These data suggest the possibility of E-selectin/AAV attenuating gangrene versus LacZ/AAV administration.

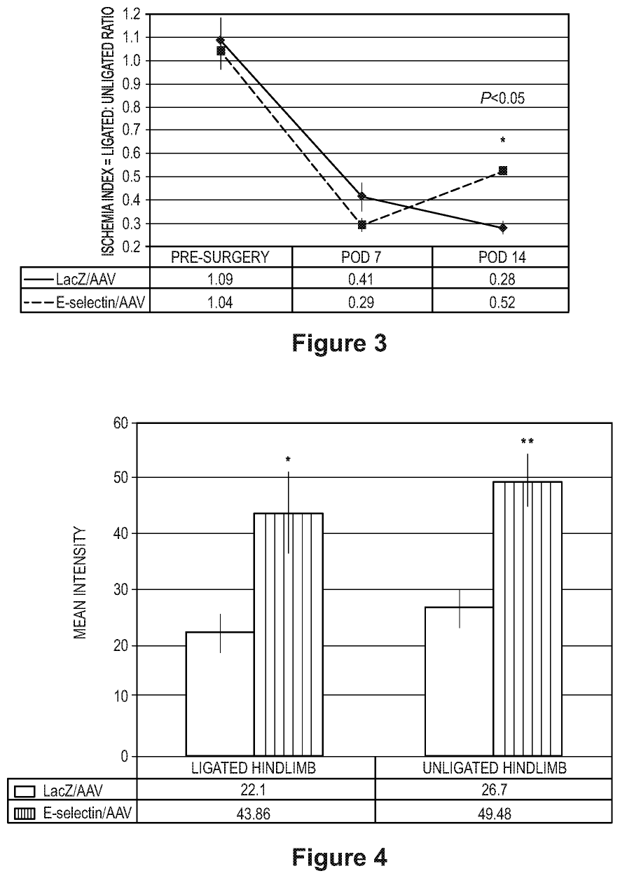

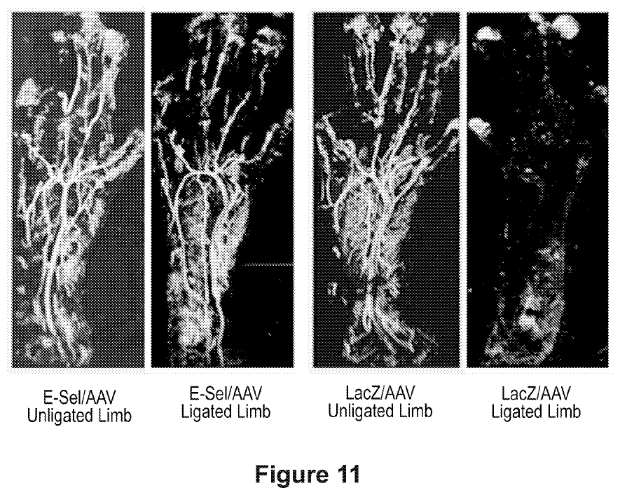

Postoperatively, all mice underwent laser Doppler imaging on days 7 and 14. By postoperative day 14 there was a significant difference in the ischemia index, calculated as a ratio of ligated to unligated limb, between groups. Where a ratio of 1 indicates normal perfusion, the ischemia index in control and treatment groups were 0.28 versus 0.52, respectively, (p<0.05) by day 14 (FIG. 3 and FIG. 10). These data confirm that the E-selectin/AAV group recovers over time with superior perfusion, and therefore less severe gangrene on gross inspection. See FIG. 11.

In addition, all mice underwent whole body Dil perfusion on postoperative day 14 before being sacrificed. Foot perfusion was detected by scanning confocal microscope. By postoperative day 14 there was a significant difference in the vessel densities of ligated foot calculated as intensity of Dil-stained blood vessels at gangrene foot in ligated versus unligated limb between groups. E-selectin/AAV gene therapy improves neovascularization and foot perfusion in gangrene foot. (FIG. 4). These data confirm that E-selectin/AAV gene therapy improves neovascularization and foot perfusion in gangrene foot.

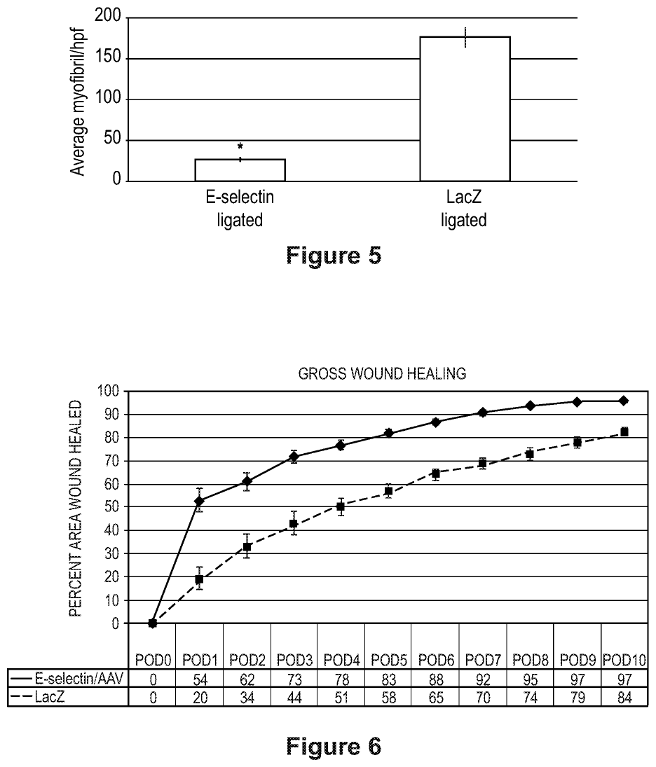

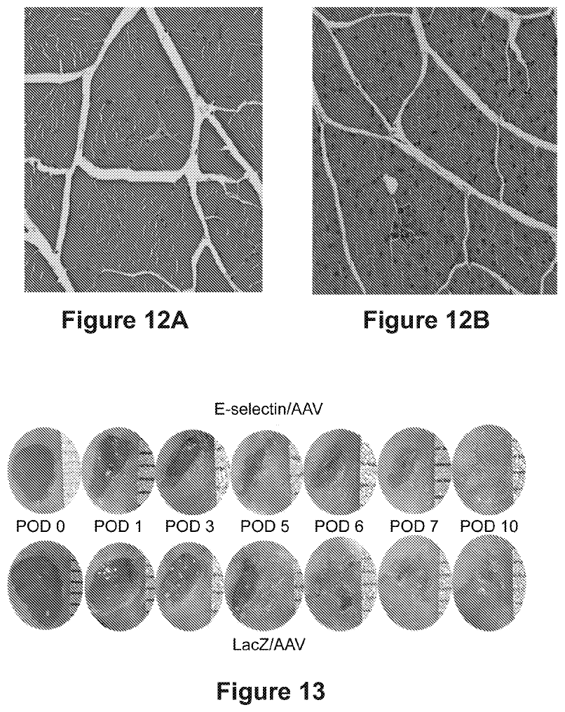

At postoperative day 14, the medial thigh muscle was harvested and fixed with formalin prior to parafinization and embedding. Based on hematoxylin and eosin staining, in the E-selectin group the ligated limb myocytes appeared less ischemic and contracted with fewer centralized nuclei, versus the LacZ ligated limb where such characteristics were more prevalently seen. Microscopic images of hematoxylin and eosin staining of E-selectin/AAV ligated and LacZ ligated limbs at 10× magnification are shown in FIGS. 12A and 12B, respectively. Higher number of myofibrils/hpf in LacZ/AAV ligated vs e-selectin ligated limb *p<0.0001. Similarly, in the LacZ/AAV group myocytes were confluent in the ligated limb with abundant inflammatory cells among centralized nuclei. Finally, the LacZ/AAV ligated group demonstrated the most number of myofibrils/hpf based on quantitative data as compared to E-selectin/AAV group (FIG. 5). These data indicate that E-selectin/AAV gene therapy improves ischemia-induced skeletal muscle atrophy.

E-selectin/AAV gene therapy also was determined to promote ischemia wound healing. FIG. 6 illustrates healing progression from POD 0-10 between E-selectin/AAV and LacZ/AAV groups. Gross images of wound healing to the inner left hindlimb thigh on postoperative days (POD) 0, 1, 3, 5, 6, 7, 10 are shown in FIG. 13. The top row of images represent images from mice treated with E-selectin/AAV and the bottom row of images represent images from mice treated with LacZ/AAV. In the E-selectin/AAV group the wound appeared to undergo the most significant contraction within the first 24 hours after surgery, progressing from 0-54% healing rate by POD1. Comparatively, LacZ/AAV mice encountered 20% healing rate by POD1 (p<0001). As mice progress from POD 0-10, E-selectin/AAV mice encountered significantly more inflammation and contraction within the wound, which is more delayed in the LacZ/AAV group based on gross images. With each day, the percent discrepancy between wound healing rates in either group lessened, yet remained statistically different. By POD 10, E-selectin/AAV mice reach 97% healing rate as compared to 84% in the LacZ/AAV group (p<0.0001).

### Example 2

This Example describes delivery of a novel stem cell-therapy in the context of the method. In one aspect, engineered BM-derived tissue repair cells (TRC) are administered, wherein E-selectin is pre-installed on the cell surface. These engineered TRC administered in ischemic limb tissue can selectively adhere and interact with activated endothelial cells (EC) (which express elevated counterpart ligands that attract and interact with E-selectin) in ischemic tissue vasculature, especially cells at the budding tip shuffling in sprouting angiogenesis, to promote new blood vessel formation. Alternatively, a supportive tissue microenvironment is generated by priming EC in wound vasculature and tissue cells with E-selectin using a viral vector. E-selectin serves as docking site for either endogenous or exogenous TRC (which express ligand of E-selectin) to anchor, by which to increase precision interaction/homing of TRC to ischemic tissue.

Intramuscular Injection of Autologous Tissue Repair Cells (TRC) Pre-Transduced with Adh/VV for Reversal of Limb Ischemia in Mouse Model.

Currently, the most widely used routes for administration of non-engineered TRC in PAD/CLI patients are intraarterial (i.a.) and intramuscular (i.m.) injection, which pose significant limitations. Intramuscularly injected TRC undergo ‘blind dating’ with tissue cells. The performance of TRC primarily relies on paracrine mechanism, since the TRC hardly and rarely interact specifically with activated EC in ischemic tissue, especially at the budding tips shuffling in sprouting angiogenesis. The intraarterial injection approach has not shown to be superior, and is accompanied by a risk of bleeding. Ischemia induced low oxygen sensor HIF-1α triggers increased expression of certain chemo-cytokines, including VEGF and SDF-1α, which not only induce angiogenesis, but also upregulate expression of a panel of adhesion molecules in endothelium of ischemic tissue. Migration and budding of tip cells is the first step of angiogenesis. Dynamic tip cell shuffling in sprouting angiogenesis is a “hot spot” to be targeted for therapeutic angiogenesis. While not wishing to be bound by any particular theory, specific interaction of TRC with multicellular budding tips will promote angiogenesis. E-selectin/ligand is a pair of adhesion molecules elevated on activated endothelium and responsible for interaction and recruitment of stem/progenitor cells, for example, endothelial progenitor cells (EPC), to the site where neovascularization occurs. It was determined that expression of E-selectin ligand is elevated in endothelium of wound tissue, likely upregulated by SDF-1α and other cytokines (i.e. IL-1 and TNF-α). To increase specific interaction of TRC with EC in budding tips, autologous LacZ+ BM-derived TRC that are pre-transduced with Adh/VV to express high levels of ‘Adh’ on cell surface will be administered (i.m.) into ischemic limb of recipient C57BL6 mice which have undergone femoral artery ligation to induce limb ischemia. The purpose to utilize LacZ+ BM-derived TRC is for easy tracking these cells in ischemic tissue post-engraftment. It was observed that wound tissue injection of autologous TRC engineered to carry E-selectin on the cell surface resulted in five-fold more increased wound angiogenesis. Engrafted TRC will more actively and specifically interact with EC in budding tips to promote angiogenesis in addition to their paracrine effect (and likely paracrine effect may be more effective due to precision attachment (close up) of TRC with target cells).

‘Priming’ Ischemic Limb Tissue Microenvironment with E-Selectin/AAV for Promoting Neo-/Re-Vascularization in a Mouse Hindlimb Gangrene Model.

Extremity gangrene is the death of leg/toe tissue and represents one of the most severe manifestations of PAD. A novel, reliable and highly reproducible model of hindlimb gangrene in the mouse is described above. Ischemic tissue vasculature and tissue cells will be primed with E-selectin by viral vector-mediated gene therapy in the mouse ischemic hindlimb gangrene model to create a highly accessible tissue microenvironment for interaction/homing of endogenous TRC. A pilot study showed that wound tissue injection of E-selectin/AAV resulted in significantly increased expression of E-selectin in wound vasculature and tissue cells in a mouse model. E-selectin expressed on vasculature and tissue cells will serve as ‘docking’ sites for endogenous TRC, which express counterpart ligand of E-selectin, to anchor and interact, which will in turn bring about a more robust revascularization and/or neovascularization response

### Methods

Construction of E-Selectin/VV and Control Vectors (Plasmids):

Viral vectors (VV) are an efficient and safe gene transfer system. Naked pDNA approach has shown limitations for the levels and duration of transgene expression. E-selectin/VV and EGFP/VV plasmids were constructed and recombinant viruses were produced. A selection marker is included in the viral vector. To this end, the basic viral vector will be modified by insertion of an IRES2-EGFP (˜1.4 kb) into multiple cloning sites. These vectors themselves can be used as controls. The murine E-selectin gene will be inserted into upstream of IRES2-EGFP using remaining sites in multiple cloning sites. Because of IRES (internal ribosome entry site), both E-selectin and marker and control genes (EGFP) can be expressed simultaneously in TRC. Thus, it will be possible to select and amplify the pure population of E-selectin-overexpressing TRC (E-selectin/TRC).

Recombinant VV (Virus) Production:

A standard method is utilized to generate recombinant VV. Recombinant VV are purified by gradient centrifugation. Fractions containing recombinant VV will be collected, dialyzed against phosphate-buffered saline (PBS), and stored.

Generation of Murine BM-Derived TRC:

Murine TRC will be generated from BM-derived mononuclear cells harvested from ROSA26 (LacZ+) mice.

Creation Ischemia-Induced Limb Gangrene in the FVB Mouse:

FVB mice are used to create ischemia-induced limb gangrene. Mice undergo unilateral femoral artery ligation and are treated with a compound that inhibits redox pathway. Incidence and progression of gangrene is assessed daily by visual inspection and photography. Gangrene is scaled using a modified Tarlov ischemia scale. Ischemia is monitored daily using LDI from post-operative day (POD) 1 to 3. Usually, mice uniformly develop distal limb gangrene by post-operative day 3.

Creation of Ischemia Limb Mouse Model in C57 BL6 Mice:

To create common ischemic limb, C57 BL6 mice undergo unilateral femoral artery and vein ligation. Hindlimb ischemia is generated by ligation of the femoral artery and vein bundle at the inguinal ligament and popliteal fossa, followed by excision of the artery/vein and all branches. Ischemia and reperfusion are monitored daily using LDI from post-operative day 1 to 7 (expected end of experiment). LDI will be performed in a temperature-controlled facility with weight-based sedation to minimize artifacts due to temperature fluctuations and level of sedation. Relative perfusion data will be expressed as the ratio of the ischemic (right) to normal (left) limb blood flow.

Transduction of Cultured TRC with Recombinant VV:

To generate E-selectin/TRC in vitro, BM-derived TRC isolated from ROSA26 (LacZ+) mice are transduced with E-selectin/VV or EGFP/VV (as control). To detect transgene (E-selectin) expression, 3-days post-transduction, TRC are detached by Trypsin-EDTA, washed and subject to FACS sorting using FACScan flow cytometer (Becton Dickinson, San Jose, Calif.) to isolate EGFP+ cells (both E-selectin/TRC and control/TRC expressing EGFP). Isolated EGFP+ cells are re-cultured for cell expansion. In addition, E-selectin expression can be validated by Western blot. TRC are rinsed with ice-cold phosphate-buffered saline (PBS) and resuspended in lysis buffer (1% Nonidit P-40, 50 mmol/1 Tris-HCl, pH 7.4, 150 mmol/1 NaCl, 200 U/ml aprotinine, 1 mmol/l PMSF). The cell lysates (10 μg of protein) were separated by 10% polyacrylamide gel electrophoresis and blotted onto polyvinylidene difluoride membranes. Immunoblotting will be performed with anti-murine E-selectin antibody and visualized with an ECL detection system.

Intramuscular Injection of E-Selectin/TRC into Ischemic Limb of Recipient C57BL6 Mouse.

Engineered TRC prepared from donor ROSA26 (LacZ+) mice are harvested and washed, re-suspended in PBS. 100 μl of cell suspension (AAV/TRC versus control/TRC: 8 mice/group) will be injected (i.m.) into ischemic limb.

Transduction of Ischemic Limb Tissue with Recombinant VV:

FVB mice are anesthetized with diethyl ether. E-selectin/VV and EGFP/VV are diluted in saline solution. The viral suspension will be injected into ipsilateral semimembranosus muscle. Transgene (E-selectin) expression will be examined by immunofluorescence (IF).

Monitor Hindlimb Ischemia or Gangrene:

Limb ischemia and restoration of blood flow into ischemic hindlimbs are monitored daily using LDI from post-operative day 1 until day 7 at a temperature-controlled facility. Relative perfusion data will be expressed as the ratio of the ischemic (right) to normal (left) limb blood flow.

Evaluation of Neovascularization and Revascularization:

Limb tissue blood vessel density will be measured by Dil perfusion and Laser scanning confocal microscopy. Mouse blood vessels (neovascularization), will be directly labeled in vivo in anesthetized mice by live perfusion using a specially formulated aqueous solution containing Dil (D-282, Invitrogen/Molecular Probes). The vascular network in limb tissue will be visualized by scanning the ischemic limb to a thickness or depth of 200 μm, using laser scanning confocal microscopy. Vessel density is quantified assessing total number of red Dil-labeled vessels normalized to the entire scanned area, using ImageJ software. In addition, angiogenesis will also be evaluated by quantification of capillary density with immunostaining for endothelial cell marker KDR or CD31 with anti-KDR or anti-CD31 antibody using harvested limb tissue. Re-vascularization is measured by both LDI (daily) and visualization of lateral femoral artery formation by Laser scanning confocal microscopy (focusing on large vessels near ligated femoral area) following Dil perfusion.

MicroCT:

Vascularization can also be explored by microCT. Micro-computed Tomography (micro-CT) provides high-resolution 3D volumetric data suitable for analysis, quantification, validation, and visualization of vascular imaging. It offers as an alternative method for vascular exploration in experimental mice.

Detection of Incorporation of Autologous E-Selectin/TRC into Tissue Endothelium:

Since administered TRC are from ROSA26(LacZ+) donor mice, E-selectin/TRC and control EGFP/TRC can be detected by X-gal staining, by which number and tissue location of LacZ+ TRC can be examined (cell tracking). Alternatively, since the TRC are EGFP+, and detectable by IF (anti-EGFP staining). X-gal can be used as it brings about more stable results with less background than immunofluorescence (IF).

β-Galactosidase Assay (X-Gal) and IF for Tissue-Level Detection of BM-Derived TRC and their Fate:

Harvested limb tissues are separated into two parts, one for freezing and one for fixation (4% paraformaldehyde). Frozen tissue sections are then incubated with X-gal (Fermentas, Canada) for 2 hours at room temperature. Sections were counterstained with nuclear fast red (Vector Labs). The number of TRC is quantified by counting β-galactosidase+ cells in serial sections of limb tissues at post-operative day 7 in 5 random high power fields (HPF, 40×) per section in at least 3 serial sections. Engrafted TRC can also be detected by IF to stain EGFP using standard protocol. To detect cell fate, HRP-conjugated antibodies for differentiation markers, for example, KDR for EC lineage and FSP-1 for fibroblasts, are added when frozen sections are incubated with X-gal. Positive staining is detected using HRP/DAB or AEC Detection IHC Kit (ab93702, Abcam). For fixed tissue section, fluorescent dye-conjugated Abs against GFP, KDR or FSP-1 can be used. Unclear is counterstained with DAPI.

Investigation of the Location and Fate of Incorporated E-Selectin/TRC in Ischemia Limb:

To detect location and fate of injected TRC, IHC is used to co-stain cell lineage marker, e.g., KDR for EC and FSP-1 for fibroblasts, with X-gal.

Profiling Cytokine/Chemokine in Recovered Ischemia Limb:

To explore potential paracrine effect of injected E-selectin/TRC on therapeutic angiogenesis, test cell and tissue lysates are tested using MILLIPLEX MAP Mouse Angiogenesis/Growth Factor Magnetic Bead Panel (Millipore), which detects 27-cytokines: Angiopoietin-2, G-CSF, sFasL, sAlk-1, Amphiregulin, Leptin, IL-1β, Betacellulin, EGF, IL-6, Endoglin, Endothelin-1, FGF-2, Follistatin, HGF, PECAM-1, IL-17, PLGF-2, KC, MCP-1, Prolactin, MIP-1α, SDF-1α, VEGF-C, VEGF-D, VEGF-A, and TNFα.

Data Analysis:

Statistical analysis of differences are performed using a 2-tailed Student's t-test for two-group comparisons and two-way ANOVA test for multiple comparisons. P-values are considered statistically significant at P<0.05. Statistical analyses will be carried out with the SPSS 22.0 software package (SPSS, Inc., Chicago, Ill., USA).

### Example 3

The lack of a reliable animal hindlimb gangrene model limits molecular investigation and pre-clinical treatment of critical limb ischemia. This example describes the development and use of a mouse hindlimb gangrene model for assessing the efficacy of gene therapy.

It was hypothesized that priming ischemic hindlimb tissue with E-selectin/adenoassociated virus (AAV) would enhance therapeutic angiogenesis and attenuate gangrene.

Two methods to induce hindlimb gangrene were tested. In a first method, FVB mice underwent femoral artery ligation (FAL) to achieve critical limb ischemia. In a second method, FVB mice underwent combined FAL and administration of NG-nitro-L-arginine methyl ester (L-NAME), a nitric oxide synthase inhibitor, which further reduces hindlimb perfusion. Prior to FAL and L-NAME use, gangrene-induced mice were intramuscularly administered E-selectin/AAV (treatment) or LacZ/AAV (control) to the hindlimb. Gangrene was assessed using a standardized ischemia score ranging from 0 (no gangrene) to 11 (forefoot gangrene), recorded on postoperative day (POD)'s 2, 7, 14. Hindlimb reperfusion using Laser doppler imaging was quantified by mean perfusion of ligated:non-ligated limb on same POD's. Live animal Dil perfusion plus laser scanning confocal microscopy was used to quantify limb neovascularization.

Most FVB did not develop gangrene with FAL-only method (n=2/8, 25% gangrene incidence). Combined FAL and L-NAME method consistently induced hindlimb gangrene (n=14/14, 100% gangrene incidence). Laser doppler imaging score on POD 7 for E-selectin/AAV (n=7) and LacZ/AAV (n=7) was 0.41 versus 0.27 (P=0.071) and on POD 14 was 0.54 versus 0.29 (P=0.017). Dil perfused ligated hindlimb in E-selectin/AAV and LacZ/AAV revealed significantly different mean neovascularization intensity score of 44 versus 21 (P=0.037). Dil perfused non-ligated limb in respective mice demonstrated mean intensity score of 50 versus 25 (P=0.006). Mean limb ischemia score on POD 2, 7, 14 for E-selectin/AAV and LacZ/AAV was 1.9, 2.9 and 3.7 versus 2.7, 3.9 and 5.3 (P=0.104).

A highly reliable mouse hindlimb gangrene model was successfully developed and shown to be useful for translational studies. Using this model, the E-selectin-based novel gene therapy improved limb reperfusion and increased neovascuclarization in critical limb ischemia. This novel hindlimb gangrene model can be utilized to further understand Redox pathways contributing to gangrene, facilitating future translational studies.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed:

1. A method of increasing blood flow or perfusion in an ischemic tissue in a subject, comprising administering to the subject in an amount effective to increase the blood flow or perfusion in the ischemic tissue a hybrid adenoassociated virus (AAV) comprising a nucleotide sequence encoding an E-selectin, AAV serotype 2 (AAV2) inverted terminal repeats (ITRs), and a capsid from an AAV other than serotype 2.

2. A method of inducing angiogenesis, neovascularization or revascularization in an ischemic tissue in a subject, comprising administering to the subject in an amount effective to induce angiogenesis, neovascularization or revascularization in the ischemic tissue a hybrid adenoassociated virus (AAV) comprising a nucleotide sequence encoding an E-selectin, AAV serotype 2 (AAV2) inverted terminal repeats (ITRs), and a capsid from an AAV other than serotype 2.

3. A method of increasing skeletal muscle viability in a subject, comprising administering to the subject in an amount effective to increase the skeletal muscle viability a hybrid adenoassociated virus (AAV) comprising a nucleotide sequence encoding an E-selectin, AAV serotype 2 (AAV2) inverted terminal repeats (ITRs), and a capsid from an AAV other than serotype 2.

4. A method of promoting ischemic skin wound healing in a subject, comprising administering to the subject in an amount effective to promote ischemic skin wound healing a hybrid adenoassociated virus (AAV) comprising a nucleotide sequence encoding an E-selectin, AAV serotype 2 (AAV2) inverted terminal repeats (ITRs), and a capsid from an AAV other than serotype 2.

5. A method of treating or preventing gangrene in a subject, comprising administering to the subject in an amount effective to treat or prevent gangrene in the subject a hybrid adenoassociated virus (AAV) comprising a nucleotide sequence encoding an E-selectin, AAV serotype 2 (AAV2) inverted terminal repeats (ITRs), and a capsid from an AAV other than serotype 2.

6. The method of claim any one of claims 1-5, wherein the subject has critical limb ischemia (CLI).

7. A method of treating critical limb ischemia (CLI) in a subject, comprising administering to the subject in an amount effective to treat CLI a hybrid adenoassociated virus (AAV) comprising a nucleotide sequence encoding an E-selectin, AAV serotype 2 (AAV2) inverted terminal repeats (ITRs), and a capsid from an AAV other than serotype 2.

8. The method of any one of claims 1-7, wherein the subject has peripheral artery disease (PAD).

9. The method of any one of claims 1-8, wherein the capsid is an AAV serotype 8 capsid and the hybrid AAV is an AAV2/8.

10. The method of any one of claims 1-8, wherein the capsid is an AAV serotype 9 capsid and the hybrid AAV is an AAV2/9.

11. The method of any one of the previous claims, wherein the AAV is intramuscularly administered to the ischemic tissue of the skeletal muscle.

12. A method of increasing blood flow or perfusion in an ischemic tissue in a subject, comprising administering to the subject a cell comprising an adenoassociated virus (AAV) comprising a nucleotide sequence encoding an E-selectin, AAV serotype 2 (AAV2) inverted terminal repeats (ITRs), and a AAV2 capsid in an amount effective to increase the blood flow or perfusion in the ischemic tissue.

13. A method of inducing angiogenesis, neovascularization or revascularization in an ischemic tissue in a subject, comprising administering to the subject a cell comprising an adenoassociated virus (AAV) comprising a nucleotide sequence encoding an E-selectin, AAV serotype 2 (AAV2) inverted terminal repeats (ITRs), and a AAV2 capsid in an amount effective to induce angiogenesis, neovascularization or revascularization in the ischemic tissue.

14. A method of promoting ischemic skin wound healing in a subject, comprising administering to the subject a cell comprising an adenoassociated virus (AAV) comprising a nucleotide sequence encoding an E-selectin, AAV serotype 2 (AAV2) inverted terminal repeats (ITRs), and a AAV2 capsid in an amount effective to promote ischemic skin wound healing.

15. A method of treating or preventing gangrene in a subject, comprising administering to the subject a cell comprising an adenoassociated virus (AAV) comprising a nucleotide sequence encoding an E-selectin, AAV serotype 2 (AAV2) inverted terminal repeats (ITRs), and a AAV2 capsid in an amount effective to treat or prevent gangrene in the subject.

16. The method of claim any one of claims 12-15, wherein the subject has critical limb ischemia (CLI).

17. A method of treating critical limb ischemia (CLI) in a subject, comprising administering to the subject a cell comprising an adenoassociated virus (AAV) comprising a nucleotide sequence encoding an E-selectin, AAV serotype 2 (AAV2) inverted terminal repeats (ITRs), and a AAV2 capsid in an amount effective to treat the CLI in the subject.

18. The method of any one of claims 12-17, wherein the subject has peripheral artery disease (PAD).

19. The method of any one claims 12-18, wherein the cell is a stem cell.

20. The method of claim 19, wherein the cell is a mesnchymal stem cell (MSC), a bone marrow (BM)-derived progenitor cell, or an endothelial progenitor cell (EPC).

21. The method of any one of claims 12-20, wherein the cell is autologous the subject.


SEQUENCE LISTING (9 sequences)

SEQ ID NO: 1 - protein, 610 aa, Homo sapiens
  ref <308>: DATABASE ACCESSION NUMBER: Genbank / NP_000441.2
  ref <309>: DATABASE ENTRY DATE: 2017-04-09
  ref <313>: RELEVANT RESIDUES IN SEQ ID NO: (1)..(610)
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 2 - DNA, 3875 bp, Homo sapiens
  ref <308>: DATABASE ACCESSION NUMBER: Genbank / NM_000450.2
  ref <309>: DATABASE ENTRY DATE: 2017-04-09
  ref <313>: RELEVANT RESIDUES IN SEQ ID NO: (1)..(3875)
agctgttcttggctgacttcacatcaaaactcctatactgacctgagacagaggcagcagtgatacccacctgagagatcctgtgtttgaacaactgcttcccaaaacggaaagtatttcaagcctaaacctttgggtgaaaagaactcttgaagtcatgattgcttcacagtttctctcagctctcactttggtgcttctcattaaagagagtggagcctggtcttacaacacctccacggaagctatgacttatgatgaggccagtgcttattgtcagcaaaggtacacacacctggttgcaattcaaaacaaagaagagattgagtacctaaactccatattgagctattcaccaagttattactggattggaatcagaaaagtcaacaatgtgtgggtctgggtaggaacccagaaacctctgacagaagaagccaagaactgggctccaggtgaacccaacaataggcaaaaagatgaggactgcgtggagatctacatcaagagagaaaaagatgtgggcatgtggaatgatgagaggtgcagcaagaagaagcttgccctatgctacacagctgcctgtaccaatacatcctgcagtggccacggtgaatgtgtagagaccatcaataattacacttgcaagtgtgaccctggcttcagtggactcaagtgtgagcaaattgtgaactgtacagccctggaatcccctgagcatggaagcctggtttgcagtcacccactgggaaacttcagctacaattcttcctgctctatcagctgtgataggggttacctgccaagcagcatggagaccatgcagtgtatgtcctctggagaatggagtgctcctattccagcctgcaatgtggttgagtgtgatgctgtgacaaatccagccaatgggttcgtggaatgtttccaaaaccctggaagcttcccatggaacacaacctgtacatttgactgtgaagaaggatttgaactaatgggagcccagagccttcagtgtacctcatctgggaattgggacaacgagaagccaacgtgtaaagctgtgacatgcagggccgtccgccagcctcagaatggctctgtgaggtgcagccattcccctgctggagagttcaccttcaaatcatcctgcaacttcacctgtgaggaaggcttcatgttgcagggaccagcccaggttgaatgcaccactcaagggcagtggacacagcaaatcccagtttgtgaagctttccagtgcacagccttgtccaaccccgagcgaggctacatgaattgtcttcctagtgcttctggcagtttccgttatgggtccagctgtgagttctcctgtgagcagggttttgtgttgaagggatccaaaaggctccaatgtggccccacaggggagtgggacaacgagaagcccacatgtgaagctgtgagatgcgatgctgtccaccagcccccgaagggtttggtgaggtgtgctcattcccctattggagaattcacctacaagtcctcttgtgccttcagctgtgaggagggatttgaattacatggatcaactcaacttgagtgcacatctcagggacaatggacagaagaggttccttcctgccaagtggtaaaatgttcaagcctggcagttccgggaaagatcaacatgagctgcagtggggagcccgtgtttggcactgtgtgcaagttcgcctgtcctgaaggatggacgctcaatggctctgcagctcggacatgtggagccacaggacactggtctggcctgctacctacctgtgaagctcccactgagtccaacattcccttggtagctggactttctgctgctggactctccctcctgacattagcaccatttctcctctggcttcggaaatgcttacggaaagcaaagaaatttgttcctgccagcagctgccaaagccttgaatcagatggaagctaccaaaagccttcttacatcctttaagttcaaaagaatcagaaacaggtgcatctggggaactagagggatacactgaagttaacagagacagataactctcctcgggtctctggcccttcttgcctactatgccagatgcctttatggctgaaaccgcaacacccatcaccacttcaatagatcaaagtccagcaggcaaggacggccttcaactgaaaagactcagtgttccctttcctactctcaggatcaagaaagtgttggctaatgaagggaaaggatattttcttccaagcaaaggtgaagagaccaagactctgaaatctcagaattccttttctaactctcccttgctcgctgtaaaatcttggcacagaaacacaatattttgtggctttctttcttttgcccttcacagtgtttcgacagctgattacacagttgctgtcataagaatgaataataattatccagagtttagaggaaaaaaatgactaaaaatattataacttaaaaaaatgacagatgttgaatgcccacaggcaaatgcatggagggttgttaatggtgcaaatcctactgaatgctctgtgcgagggttactatgcacaatttaatcactttcatccctatgggattcagtgcttcttaaagagttcttaaggattgtgatatttttacttgcattgaatatattataatcttccatacttcttcattcaatacaagtgtggtagggacttaaaaaacttgtaaatgctgtcaactatgatatggtaaaagttacttattctagattaccccctcattgtttattaacaaattatgttacatctgttttaaatttatttcaaaaagggaaactattgtcccctagcaaggcatgatgttaaccagaataaagttctgagtgtttttactacagttgttttttgaaaacatggtagaattggagagtaaaaactgaatggaaggtttgtatattgtcagatattttttcagaaatatgtggtttccacgatgaaaaacttccatgaggccaaacgttttgaactaataaaagcataaatgcaaacacacaaaggtataattttatgaatgtctttgttggaaaagaatacagaaagatggatgtgctttgcattcctacaaagatgtttgtcagatatgatatgtaaacataattcttgtatattatggaagattttaaattcacaatagaaactcaccatgtaaaagagtcatctggtagatttttaacgaatgaagatgtctaatagttattccctatttgttttcttctgtatgttagggtgctctggaagagaggaatgcctgtgtgagcaagcatttatgtttatttataagcagatttaacaattccaaaggaatctccagttttcagttgatcactggcaatgaaaaattctcagtcagtaattgccaaagctgctctagccttgaggagtgtgagaatcaaaactctcctacacttccattaacttagcatgtgttgaaaaaaaagtttcagagaagttctggctgaacactggcaacaacaaagccaacagtcaaaacagagatgtgataaggatcagaacagcagaggttcttttaaaggggcagaaaaactctgggaaataagagagaacaactactgtgatcaggctatgtatggaatacagtgttattttctttgaaattgtttaagtgttgtaaatatttatgtaaactgcattagaaattagctgtgtgaaataccagtgtggtttgtgtttgagttttattgagaattttaaattataacttaaaatattttataatttttaaagtatatatttatttaagcttatgtcagacctatttgacataacactataaaggttgacaataaatgtgcttatgttta

SEQ ID NO: 3 - protein, 619 aa, Mus musculus
  ref <308>: DATABASE ACCESSION NUMBER: Genbank / AAA37577.1
  ref <309>: DATABASE ENTRY DATE: 2005-10-07
  ref <313>: RELEVANT RESIDUES IN SEQ ID NO: (1)..(619)
MKATAGVMNASRFLSALVFVLLAGESTAWYYNASSELMTYDEASAYCQRDYTHLVAIQNKEEINYLNSNLKHSPSYYWIGIRKVNNVWIWVGTGKPLTEEAQNWAPGEPNNKQRNEDCVEIYIQRTKDSGMWNDERCNKKKLALCYTASCTNASCSGHGECIETINSYTCKCHPGFLGPNCEQAVTCKPQEHPDYGSLNCSHPFGPFSYNSSCSFGCKRGYLPSSMETTVRCTSSGEWSAPAPACHVVECEALTHPAHGIRKCSSNPGSYPWNTTCTFDCVEGYRRVGAQNLQCTSSGIWDNETPSCKAVTCDAIPQPQNGFVSCSHSTAGELAFKSSCNFTCEQSFTLQGPAQVECSAQGQWTPQIPVCKAVQCEALSAPQQGNMKCLPSASGPFQNGSSCEFSCEEGFELKGSRRLQCGPRGEWDSKKPTCSAVKCDDVPRPQNGVMECAHATTGEFTYKSSCAFQCNEGFSLHGSAQLECTSQGKWTQEVPSCQVVQCPSLDVPGKMNMSCSGTAVFGTVCEFTCPDDWTLNGSAVLTCGATGRWSGMPPTCEAPVSPTRPLVVALSAAGTSLLTSSSLLYLLMRYFRKKAKKFVPASSCQSLQSFENYHVPSYNV

SEQ ID NO: 4 - protein, 549 aa, Rattus norvegicus
  ref <308>: DATABASE ACCESSION NUMBER: Genbank / AAA41113
  ref <309>: DATABASE ENTRY DATE: 1993-10-21
  ref <313>: RELEVANT RESIDUES IN SEQ ID NO: (1)..(549)
MNASCFLSALTFVLLIGKSIAWYYNASSELMTYDEASAYCQRDYTHLVAIQNKEEINYLNSTLRYSPSYYWIGIRKVNNVWIWVGTQKPLTEEAKNWAPGEPNNKQRNEDCVEIYIQRPKDSGMWNDERCDKKKLALCYTASCTNTSCSGHGECVETINSYTCKCHPGFLGPKCDQVVTCQEQEYPDHGSLNCTHPFGLFSYNSSCSFSCERGYVPSSMETTVRCTSSGEWSAPAPACHVVECKALTQPAHGVRKCSSNPGSYPWNTTCTFDCEEGYRRVGAQNLQCTSSGVWDNEKPSCKAVTCDAIPRPQNGSVSCSNSTAGALAFKSSCNFTCEHSFTLQGPAQVECSAQGQWTPQIPVCKASQCEALSAPQRGHMKCLPSASAPFQSGSSCKFSCDEGFELKGSRRLQCGPRGEWDSEKPTCAGVQCSSLDLPGKMNMSCSGPAVFGTVCEFTCPEGWTLNGSSILTCGATGRWSAMLPTCEAPANPPRPLVVALSVAATSLLTLSSLIYVLKRFFWKKAKKFVPASSCQSLQSFENYQGPSYII

SEQ ID NO: 5 - protein, 611 aa, Canis lupus
  ref <308>: DATABASE ACCESSION NUMBER: Genbank / AAA30843
  ref <309>: DATABASE ENTRY DATE: 1993-08-13
  ref <313>: RELEVANT RESIDUES IN SEQ ID NO: (1)..(611)
MITSQLLPALTLVLLLFKEGGAWSYNASTEAMTFDEASTYCQQRYTHLVAIQNQEEIKYLNSMFTYTPTYYWIGIRKVNKKWTWIGTQKLLTEEAKNWAPGEPNNKQNDEDCVEIYIKRDKDSGKWNDERCDKKKLALCYTAACTPTSCSGHGECVETVNNYTCKCHPGFRGLRCEQVVTCQAQEAPEHGSLVCTHPLGTFSYNSSCFVSCDKGYLPSSTEATQCTSTGEWSASPPACNVVECSALTNPCHGVMDCLQSSGNFPWNMTCTFECEEGFELMGPKRLQCTSSGNWDNRKPTCKAVTCGAIGHPQNGSVSCSHSPAGEFSVRSSCNFTCNEGFLMQGPAQIECTAQGQWSQQVPVCKASQCKALSSPERGYMSCLPGASGSFQSGSSCEFFCEKGFVLKGSKTLQCGLTGKWDSEEPTCEAVKCDAVQQPQDGLVRCAHSSTGEFTYKSSCAFSCEEGFELHGSAQLECTSQGQGVTGGPSCQVVQCFKSGSFRKDEHKLQGEPVFGAVCAFACPEGWTLNGSAALMCDATGHWSGMLPTCEAPTESSIPLAVGLTAGGTSLLTVASFLLWLLKRLRKRAKKFVPASSCQSLQSDGSYHMPCSI

SEQ ID NO: 6 - protein, 484 aa, Ovis aries
  ref <308>: DATABASE ACCESSION NUMBER: Genbank / NP_001009749
  ref <309>: DATABASE ENTRY DATE: 2013-04-18
  ref <313>: RELEVANT RESIDUES IN SEQ ID NO: (1)..(484)
MIASQYLSALTFVLLFKESRTWSYHASTEIMTFEAARDYCQKTYTALVAIQNQEEIEYLNSTFSHSPSYYWIGIRKINGTWTWIGTNKSLTEEATNWAPGEPNNKQTDEDCVEIYIKREKDSGKWNDENCTKKKFALCYKAACTPTSCSSHGECVETINSHTCQCHPGFKGLRCEQVVTCQAQKHPEHGHLVCNPLGKFTYNSSCSISCAEGYLPSSTEAARCMSSGEWSTPLPKCNVVKCDALSNPDNGVVNCSQNHGSLPWNTTCMFECQEGYKLTGPQHLQCTSSGIWDNKQPTCKAVTCAAISHPQNGTVKCSHSLVGDFAFKSSCHFTCAEGFALQGPAQVECTAQGRWTQQVPVCEVVRCSRLDVSGKLNMDCNGEPVLGTECMFACPEGWTLNGSVALTCGATGHWSGMLPTCEAPTVSQTPLAVGLSTAGVSLVTIPSFLFWLLKRLQMKAEKLSDDSSCSSLKSNGCYSTPSELV

SEQ ID NO: 7 - protein, 21 aa, Homo sapiens
MIASQFLSALTLVLLIKESGA

SEQ ID NO: 8 - protein, 589 aa, Homo sapiens
  MISC_FEATURE: Mature E-selectin
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 9 - DNA, 1860 bp, Mus musculus
atgaaagcaactgctggagtcatgaatgcctcgcgctttctctctgctcttgtttttgttctcctcgctggagagagcacagcttggtactacaatgcctccagtgagctcatgacgtatgatgaagccagtgcatactgtcagcgggactacacacatctggtggcgattcagaacaaggaagagatcaactaccttaactccaatctgaaacattcaccgagttactactggattggaatcagaaaagtcaataacgtatggatctgggtggggacggggaagcctctgacagaggaagctcagaactgggctccaggtgaaccaaacaacaaacaaagaaatgaggactgtgtagagatttacatccaacgaaccaaagactcgggcatgtggaatgacgagagatgtaacaaaaagaagctggctctgtgctacacagcttcgtgtaccaatgcatcctgcagtggtcatggtgaatgcatagagaccatcaatagttacacctgcaagtgccaccctggcttcctgggacccaactgtgagcaagctgtgacttgcaaaccacaggaacaccctgactatggaagcctgaactgctcccacccgttcggccccttcagctataattcctcctgctcctttggctgtaaaaggggctacctgcccagcagcatggagaccaccgtgcggtgtacgtcctctggagagtggagtgcgcctgctccagcctgccatgtggttgaatgtgaagctttgacccaccctgcccacggtatcaggaaatgttcctcaaatcctgggagctacccatggaacacgacatgcacgtttgactgtgtggaagggtacaggcgagttggagctcagaatctacagtgtacctcatctggcatctgggataacgagacgccatcatgcaaagctgtgacctgtgacgccatccctcagcctcagaatggctttgtgagctgcagccactcaacagctggagaacttgcgtttaagtcatcctgtaacttcacctgtgagcagagtttcacgttgcaggggccagcgcaggttgaatgcagcgcacaagggcagtggacaccacaaatcccagtctgcaaagctgtccagtgtgaagccttatctgcgccacagcagggcaacatgaaatgtcttcccagtgcttctggacctttccaaaatgggtccagttgtgagttctcctgcgaagaaggatttgaactgaagggatcaagaagacttcagtgtggtccaagaggggaatgggatagcaagaagcccacgtgttcagctgtgaaatgtgatgatgtccctcggccccagaatggcgtcatggagtgtgctcatgctactactggagaattcacctacaagtcctcatgtgcctttcaatgcaatgagggctttagcttgcatggctcagctcaacttgagtgcacatctcagggaaagtggacccaggaagtcccctcctgccaagtggtacaatgtccaagccttgacgtcccgggaaagatgaacatgagctgcagcggaacagcagttttcggcacagtgtgtgagtttacatgtcctgatgattggacactcaatggatctgcagttctgacgtgtggtgccacgggacgctggtctgggatgccgcctacctgtgaagccccagtcagccccacccgtcccttggtagttgcactttctgcggcaggaacctcactcctgacatcgtcctcattgctctacttgttgatgagatactttcggaagaaagcaaagaaatttgttcctgctagcagctgccaaagccttcaatcatttgaaaactaccatgtgccttcttacaacgtctag